(19) United States
(12) Patent Application Publication
(10) Pub. No.: US 2012/0302843 A1
(43) Pub. Date: Nov. 29, 2012

(54) HEALTH MANAGEMENT SUPPORT DEVICE, HEALTH MANAGEMENT SUPPORT SYSTEM, AND HEALTH MANAGEMENT SUPPORT PROGRAM

(75) Inventors: Yutaka OTSUBO, Takatsuki-shi (JP); Akiyoshi FUJISAKI, Kyoto-shi (JP)

(73) Assignee: OMRON HEALTHCARE CO., LTD., Muko-shi (JP)

(21) Appl. No.: 13/572,182

(22) Filed: Aug. 10, 2012

Related U.S. Application Data
(63) Continuation of application No. PCT/JP2011/054593, filed on Mar. 1, 2011.

(30) Foreign Application Priority Data
Mar. 29, 2010 (JP) 2010-075331

(51) Int. Cl. A61B 5/107

(52) U.S. Cl. 600/301

(57) ABSTRACT

A health management support system includes: a unit that receives two or more types of physical information measured by a user along with measurement time data; an analyzing unit for analyzing the relationship between the received two or more types of physical information in accordance with a predetermined rule; an advice generating unit that generates advice based on a result of the analysis; and an advice output unit that outputs the generated advice. The analyzing unit has a knowledge file that stores the predetermined rule, and an engine unit for executing the analysis. The advice generating unit generates the advice for notifying the user of a goal achievement level by analyzing the two or more types of physical information measured in a first predetermined period.

### TECHNICAL FIELD

This invention relates to health management support devices, health management support systems, and health management support programs, and particularly relates to a health management support device, a health management support system, and a health management support program that analyze physical information and information regarding lifestyle factors collected from a user and provide health management advice based on results of the analysis.

### BACKGROUND ART

Recent years have seen an increasing trend toward health awareness, and more attention is being given to techniques that use a server device to analyze information uploaded from healthcare devices connected to networks, day-to-day lifestyle records inputted over the Internet, and so on, and navigate (guide; advise) users' behavior with respect to their health.

In systems that support a user in managing his or her health by him or herself, providing specific methods and techniques for improving lifestyle habits is extremely important as a way to modify the user's behavior. In conventional fully-automatic health management support systems, in which users were not involved, users were supported through functions such as periodic questions and advice, user searches for healthcare information, monitoring of healthcare device data and the like (records, graph displays), and so on; however, there have been difficulties in terms of accuracy issues caused by the manual input of measured data, infrequent support intervention on the part of the system, limits on providing information tailored to individuals, and the appropriate timing of behavior modification support in which the user data is applied.

(1) Providing an estimated change pattern based on bio-information analysis (JP 2005-319283A) has been proposed as a conventional system and method for providing advice by utilizing data. (2) A system has also been proposed in which the results of health-improvement activities based on nutrition management information, health food, and so on presented to users can be checked through time-series data analysis of daily healthcare information (JP 2006-244018A). (3) Furthermore, a system and method have been proposed in which advice from a doctor is followed up on by monitoring trends (inclinations) in the average values of lifestyle habit data from a set period, evaluating the patient's awareness of improving his or her habits, and offering advice (JP 2007-34744A).

Meanwhile, in body weight management, which is of high interest from the standpoint of health, a method is generally known, as a morning/evening diet method, in which weight loss support is offered by finding patterns in “weight that increases from morning to evening” and “weight that decreases from evening to morning” obtained from morning and evening weight measurements, and supporting weight loss based on the frequencies at which such patterns appear. Typical weight loss support that uses morning/evening body weight differences handles the “weight that increases from morning to evening” and “weight that decreases from evening to morning” based on a reference of approximately 500 to 600 g from an empirical value or approximately 0.7% of the current body weight.

As a method for displaying an intra-day body weight change by measuring a body weight using a conventional scale or body composition meter, (1) a method has been proposed in which reference data used for comparison is created from body weight measurement times and past data measured at those times, and the result of comparison with that reference data is displayed, or alternatively, whether the body weight is in an increasing trend or a decreasing trend is displayed through comparison with intra-day fluctuations occurring on the day of measurement (JP 2005-218582A). (2) In addition, a method has been proposed in which it is determined whether or not a fluctuation range for a body weight measurement value within the same day is within a predetermined reference range, and the percentage of days in which a positive determination is made within a predetermined period is displayed (JP 2008-304421).

Furthermore, as a weight loss support method for daily body weight input in a conventional instruction support system, (3) a method has been proposed in which weight loss is estimated, periods of no weight loss are detected, and so on and advice is given, based on the degree to which daily weights and daily amounts of energy increase/decrease that are inputted, as well as a change in weight from a reference date, match a pattern (JP 2008-33909A).

### CITATION LIST

### Patent Literature

Patent Literature 1: JP 2005-319283A
Patent Literature 2: JP 2006-244018A
Patent Literature 3: JP 2007-34744A
Patent Literature 4: JP 2005-218582A
Patent Literature 5: JP 2008-304421A
Patent Literature 6: JP 2008-33909A

### SUMMARY OF INVENTION

### Technical Problem

In such conventional behavior modification support for improving lifestyle habits, problems still remain, such as that the systems do not provide information in an active manner, the advice only provokes the user's attention and does not lead to behavior modification, the data analysis is generic (time-based), the timing of the advice does not take the user's convenience into consideration, and so on.

Furthermore, with body weight management that uses a conventional scale or body composition meter, the data management does not use patterns of fluctuations in one's own body weight as a base, patterns of fluctuations in one's own weight cannot be checked even if such data is used, daily body weight fluctuations cannot be viewed in relation to lifestyle cycles in a set period (one week or the like), and so on; thus there is still little motivation to lose or control one's weight.

Further still, with the stated conventional techniques, the configurations do not have procedures (rules) for analyzing physical information such as body weight provided independently from a unit that executes the analysis process by referring to such procedures, and thus updates (additions/changes) cannot be made to only the analysis procedures; it is thus not easy to modify the procedures for analysis.

Accordingly, it is an object of this invention to provide a health management support device, a health management support system, and a health management support program that provide motivation to perform healthy activities.

It is another object of this invention to provide a health management support device, a health management support system, and a health management support program that enable analysis procedures to be modified with ease.

### Solution to Problem

According to an aspect of this invention, a health management support device includes: a receiving unit that receives two or more types of physical information measured by a user along with measurement time data; an analyzing unit for analyzing the relationship between the received two or more types of physical information in accordance with a predetermined rule; an advice generating unit that generates advice based on a result of the analysis; an advice output unit that outputs the generated advice; a receiving unit that receives body weight data of the user along with measurement time data; a determination unit that determines, based on the measurement time data, whether or not the body weight data is body weight data measured during a morning time period or an evening time period; and a calculation unit that calculates, according to time series, a morning/evening body weight change amount over a set period for the body weight data determined by the determination unit to have been measured during the morning time period or the evening time period. The health management support device outputs a “body weight that increases from morning to evening” and a “body weight that decreases from evening to morning” as a graph based on the morning/evening body weight change amount during the set period calculated by the calculation unit. The analyzing unit has a knowledge file that stores the predetermined rule, and an engine unit for executing the analysis. The advice generating unit generates the advice for notifying the user of a goal achievement level by analyzing the two or more types of physical information measured in a first predetermined period.

Preferably, the health management support device generates the advice for enabling the user to achieve a goal by analyzing the two or more types of physical information measured in a second predetermined period.

Preferably, the analyzing unit analyzes changes over time in the two or more types of physical information in each of predetermined measurement periods.

Preferably, the predetermined measurement period includes a daily basis, a weekly basis, or a monthly basis.

Preferably, the advice generating unit generates advice corresponding to points in the changes over time analyzed by the analyzing unit.

Preferably, the advice generating unit generates advice corresponding to a predetermined characteristic detected over time and analyzed by the analyzing unit.

Preferably, the analyzing unit analyzes, in accordance with a predetermined rule, the two or more types of physical information and a different type of information than the physical information for a relationship between the two or more types of physical information and the different type of information than the physical information.

Preferably, the health management support device further includes: a receiving unit that receives body weight data of the user along with measurement time data; a determination unit that determines, based on the measurement time data, whether or not the body weight data is body weight data measured during a morning time period or an evening time period; a calculation unit that calculates, according to time series, a morning/evening body weight change amount over a set period for the body weight data determined by the determination unit to have been measured during the morning time period or the evening time period; a predetermined advice generating unit that generates predetermined advice based on a result of the calculation; and an advice output unit that outputs the generated predetermined advice.

Preferably, the calculation unit totals the morning/evening body weight change amount for each day of the week.

Preferably, the calculation unit calculates a variation in the morning/evening body weight change amount.

Preferably, the health management support device creates a frequency distribution for a “body weight that increases from morning to evening” and a “body weight that decreases from evening to morning” based on the morning/evening body weight change amount that is based on the body weight data measured during the set period, and outputs the frequency distribution.

Preferably, the health management support device creates a frequency distribution for a “body weight that increases from morning to evening” and a “body weight that decreases from evening to morning” based on the morning/evening body weight change amount that is based on the body weight data measured during the set period, and displays the frequency distribution as a graph.

A health management support system according to another aspect of the invention includes a server device and an information terminal. The information terminal sends two or more types of physical information measured for a user to the server device along with measurement time data and outputs information received from the server device.

The server device includes: a receiving unit that receives, from the information terminal, the two or more types of physical information along with the measurement time data; an analyzing unit for analyzing the relationship between the received two or more types of physical information in accordance with a predetermined rule; an advice generating unit that generates advice based on a result of the analysis; a sending unit that sends the generated advice to the information terminal; a receiving unit that receives body weight data of the user along with measurement time data; a determination unit that determines, based on the measurement time data, whether or not the body weight data is body weight data measured during a morning time period or an evening time period; and a calculation unit that calculates, according to time series, a morning/evening body weight change amount over a set period for the body weight data determined by the determination unit to have been measured during the morning time period or the evening time period. A “body weight that increases from morning to evening” and a “body weight that decreases from evening to morning” are outputted as a graph based on the morning/evening body weight change amount during the set period calculated by the calculation unit. The analyzing unit has a knowledge file that stores the predetermined rule, and an engine unit for executing the analysis. The advice generating unit generates the advice for notifying the user of a goal achievement level by analyzing the two or more types of physical information measured in a first predetermined period.

Preferably, the advice generating unit generates the advice for enabling the user to achieve a goal by analyzing the two or more types of physical information measured in a second predetermined period.

Preferably, the health management support system further includes one or more healthcare devices for measuring the two or more types of physical information for the user.

A health management support program according to another aspect of this invention is a health management support program that processes two or more types of physical information measured for a user, the program causing a computer to execute: a step of receiving the two or more types of physical information along with measurement time data; a step of analyzing the relationship between the received two or more types of physical information in accordance with a predetermined rule; a step of generating advice based on a result of the analysis; a step of outputting the generated advice; a step of receiving body weight data of the user along with measurement time data; a step of determining, based on the measurement time data, whether or not the body weight data is body weight data measured during a morning time period or an evening time period; and a step of calculating, according to time series, a morning/evening body weight change amount over a set period for the body weight data determined in the step of determining to have been measured during the morning time period or the evening time period. A “body weight that increases from morning to evening” and a “body weight that decreases from evening to morning” are outputted as a graph based on the morning/evening body weight change amount during the set period calculated in the step of calculating. In the step of analyzing, a knowledge file that stores the predetermined rule is referred to and the analysis is executed. In the step of generating the advice, the advice for notifying the user of a goal achievement level is generated by analyzing the two or more types of physical information measured in a first predetermined period.

Preferably, in the step of generating the advice, the advice for notifying the user of a goal achievement level is generated by analyzing the two or more types of physical information measured in a second predetermined period.

### Advantageous Effects of Invention

According to this invention, two or more types of physical information measured for a user are analyzed based on a relationship between the information, advice for notifying the user of a goal achievement level is generated based on a result of the analysis by analyzing the two or more types of physical information measured in a first predetermined period, and the advice is outputted; accordingly, healthy activities can be proposed at appropriate timings.

In addition, a knowledge file that stores predetermined rules referred to for analysis is provided independent from the engine unit that executes the analysis, and thus the predetermined rules can be updated (modified; added) independent from the engine unit. As a result, a procedure for analysis carried out for health management support can be modified with ease.

### BRIEF DESCRIPTION OF DRAWINGS

FIG. 1 is a general schematic diagram illustrating a health management support system according to an embodiment of the present invention.

FIG. 2 is a diagram illustrating the functional configuration of a server device.

FIG. 3 is a diagram schematically illustrating stored data in a data accumulation unit.

FIG. 4 is a diagram illustrating types of databases stored in the data accumulation unit.

FIG. 5 is a diagram illustrating an example of the content of a user profile database.

FIG. 6 is a diagram illustrating an example of the content of a pedometer database.

FIG. 7 is a diagram illustrating an example of the content of a body composition meter database.

FIG. 8 is a diagram illustrating an example of the content of a sphygmomanometer database.

FIG. 9 is a diagram illustrating the hardware configuration of the server device.

FIG. 10 is a diagram illustrating the hardware configuration of an information terminal.

FIG. 11 is a block diagram illustrating the configuration of a healthcare device.

FIG. 12 is a diagram illustrating a functional configuration for generating a message.

FIG. 13 is a diagram illustrating an example of variables defined by variable definition information.

FIG. 14 is a diagram illustrating an example of a message generation rule group that incorporates preliminary calculation formula information.

FIG. 15 is a diagram illustrating an example of an inputted data set.

FIG. 16 is a flowchart illustrating a measurement process executed by a scale/body composition meter.

FIG. 17 is a flowchart illustrating operations performed by a health management support system according to an embodiment of the present invention.

FIG. 18 is a diagram illustrating an example of a menu screen displayed by the health management support system.

FIG. 19 is a diagram illustrating an example of a data transfer screen displayed by the health management support system.

FIG. 20 is a diagram illustrating an example of content stored in an information terminal.

FIG. 21A is a diagram illustrating an example of details of an analysis performed on a user's physical information according to an embodiment.

FIG. 21B is a diagram illustrating an example of details of an analysis performed on a user's physical information according to an embodiment.

FIG. 22 is a diagram illustrating an example of details of an analysis performed on a user's physical information according to an embodiment.

FIG. 23A is a diagram illustrating an example of details of an analysis performed on a user's physical information according to an embodiment.

FIG. 23B is a diagram illustrating an example of details of an analysis performed on a user's physical information according to an embodiment.

FIG. 23C is a diagram illustrating an example of details of an analysis performed on a user's physical information according to an embodiment.

FIG. 24 is a process flowchart illustrating a morning/evening diet program according to an embodiment.

FIG. 25A is a diagram illustrating an example of a graph and a message displayed based on a morning/evening body weight change amount.

FIG. 25B is a diagram illustrating an example of a graph and a message displayed based on a morning/evening body weight change amount.

FIG. 26 is a diagram illustrating an example of a graph and a message displayed based on a morning/evening body weight change amount.

FIG. 27 is a diagram illustrating a histogram of a daytime weight increase occurrence frequency and a nighttime weight increase occurrence frequency.

FIG. 28 is a diagram illustrating a histogram of a daytime weight increase occurrence frequency and a nighttime weight increase occurrence frequency.

FIG. 29 is a diagram illustrating a histogram specifying frequencies of daytime weight increases and nighttime weight increases.

FIG. 30 is a diagram illustrating the frequency of appearances of body weight change amounts on a day-of-the-week basis.

FIG. 31 is a diagram illustrating the frequency of appearances of maximum, minimum, and average body weight change amounts on a day-of-the-week basis.

FIG. 32 is a graph illustrating changes in the measured values of body weight and skeletal muscle percentage along with approximated straight lines.

FIG. 33 is a diagram illustrating an average increase/decrease amount in morning body weight on a day-of-the-week basis.

FIG. 34 is a graph illustrating, over time, a cumulative value of an increase/decrease amount in morning body weight.

FIG. 35 is a graph plotting, in time series, calculated values in which one week's worth of body weight data has been smoothed.

FIG. 36A is a diagram illustrating messages displayed as a list.

FIG. 36B is a diagram illustrating messages displayed as a list.

FIG. 37A is a diagram illustrating messages displayed as a list.

FIG. 37B is a diagram illustrating messages displayed as a list.

### DESCRIPTION OF EMBODIMENTS

Hereinafter, an embodiment of this invention will be described in detail with reference to the drawings. Note that in the drawings, like reference numerals indicate like or corresponding elements, and descriptions thereof will not be repeated.

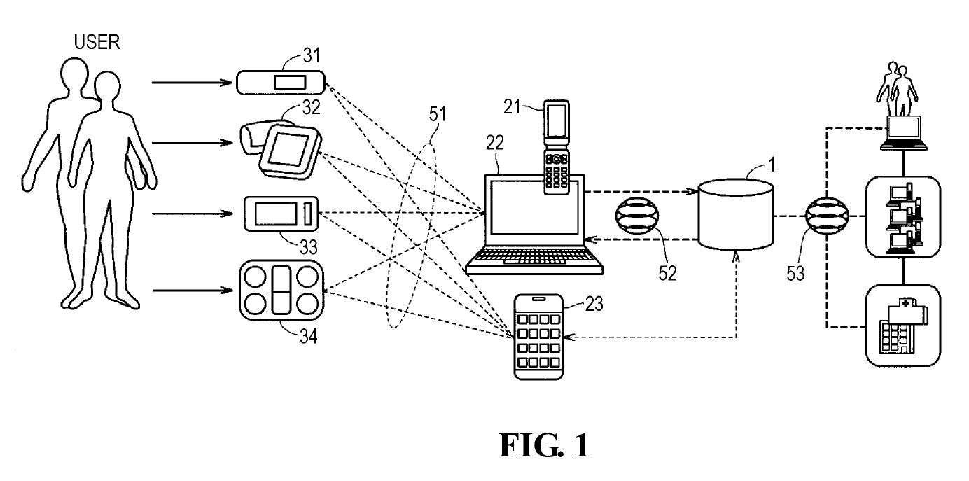

FIG. 1 is a general schematic diagram illustrating a health management support system according to an embodiment of the present invention. The health management support system measures and collects physical information in order to understand a user's lifestyle patterns and physical state of health, and to that end, includes healthcare devices worn or carried by users, information terminals 21, 22, and 23 that serve as user terminals that communicate with the healthcare devices, a server device 1 corresponding to a health management support device that communicates with the information terminals, and communication paths (communication lines) 51, 52, and 53 for connecting these devices through communications. The healthcare devices include, for example, a pedometer 33 and a sleep monitor 31 for measuring lifestyle patterns, and a scale/body composition meter 34 and a sphygmomanometer 32 for understanding a physical state of health. The healthcare devices are not limited to these types of devices.

Note that information may be exchanged among the devices using recording media instead of the communication paths 51 through 53.

The communication path 51 for connecting the healthcare devices 31 through 34 with the information terminals 21 through 23 includes a wired or wireless communication path. Short-range wireless (USB (Universal Serial Bus), BT (Bluetooth)), a contactless communication system such as FeliCa, and so on can be given as examples of wireless communication path. The communication path 52 for connecting the server device 1 with the information terminals 21 through 23, and the communication path 53 for connecting the server device 1 with a user's family's information terminal, other user information terminals, information terminals in a hospital, an exercise gym, or the like, include various types of networks, such as the Internet. The information terminals 21 through 23 include mobile or desktop-based computers having communication functions, such as users' mobile telephone terminals, PDAs (Personal Digital Assistants), personal computers, and so on. The information terminals 21 through 23 may be of any type that have functions for communicating with the server device 1 and with the healthcare devices, and are not limited to the stated types.

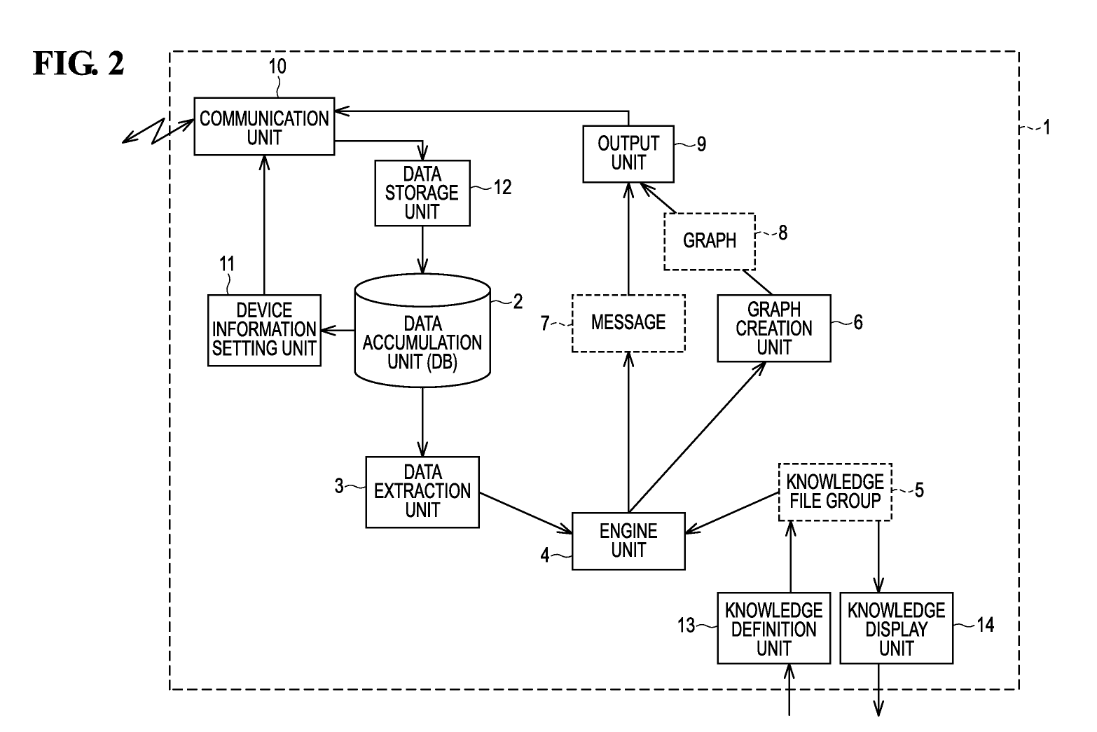

The functional configuration of the server device 1 will now be described with reference to FIG. 2. The server device 1 includes: a data accumulation unit 2, which is one type of storage unit configured of a database (DB); a data extraction unit 3 that searches out data in the data accumulation unit 2; an engine unit 4 that analyses the data searched out by the data extraction unit 3 and generates information (a message 7, a graph 8, and so on) for proposing, to a user, health-related activities based on a result of the analysis; and a knowledge file group 5 referred to by the engine unit 4.

Furthermore, the server device 1 includes: a graph creation unit 6 that creates a graphs based on data outputted from the engine unit 4; and an output unit 9 that outputs data of the graph 8 created by the graph creation unit 6 and of the message 7 outputted from the engine unit 4 to a display unit and printing unit (not shown) and a communication unit 10.

Furthermore, the server device 1 includes a data storage unit 12, for storing data received by the communication unit 10 from the information terminals 21 through 23 in the data accumulation unit 2, and a device information setting unit 11. The device information setting unit 11 takes, as its input, destination specification information specifying a destination of data read out from the data accumulation unit 2, and outputs the information to the communication unit 10. The communication unit 10 adds the destination specification information inputted from the device information setting unit 11 to data that is to be sent, such as the message 7 or graph 8 provided by the output unit 9, and sends the resulting data to the various devices, such as the information terminals 21, 22, and 23.

Furthermore, the server device 1 includes: a knowledge definition unit 13 for setting, updating, and deleting knowledge data in the knowledge file group 5 based on information from the exterior; and a knowledge display unit 14 for displaying knowledge data read out from the knowledge file group 5 to the exterior.

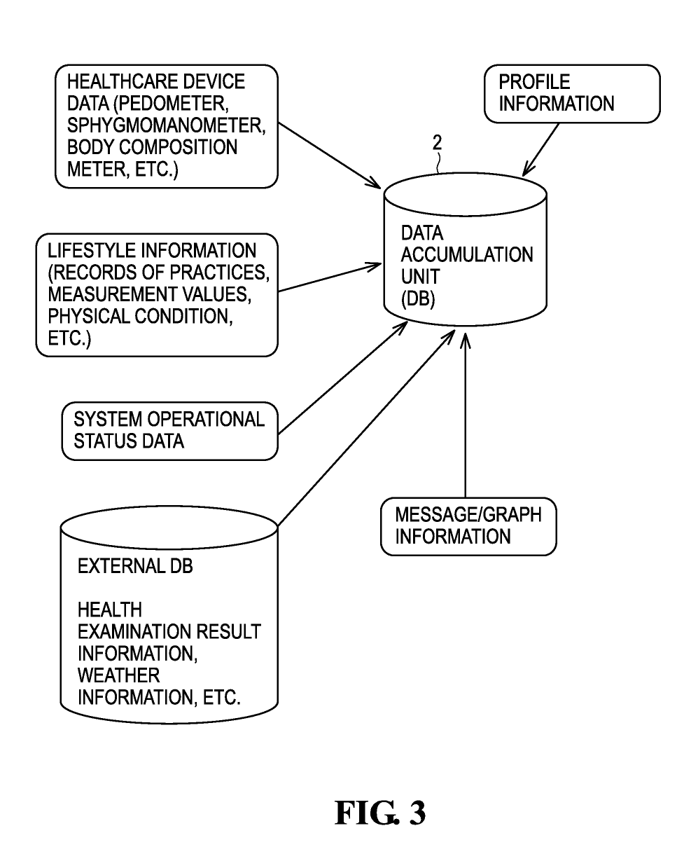

FIG. 3 schematically illustrates stored data in the data accumulation unit 2. The data stored in the data accumulation unit 2 includes: profile information of the users of the health management support system; healthcare device data, user lifestyle information, and system operational status data collected (received) from the healthcare devices 31 through 34; information of the message 7 and graph 8 generated through the analysis performed by the engine unit 4; and information (information from physical exam results, weather information, or the like) obtained from the exterior, such as from an external DB (not shown).

“Lifestyle information” refers to a user's daily practices, or information such as records, moods and physical conditions, meals/exercise/sleep/smoking, alcohol consumption, and so on that cannot be communicated with information terminals, or in other words, that is obtained through manual input of healthcare device data that is not IT (Information Technology)-based. “Profile information” includes information such as a user's nickname, sex, age, family structure, and so on.

“Healthcare device data” includes information measured by the pedometer 33 (date, number of steps, number of steps in different time periods, and so on), information measured by the sphygmomanometer 32 (systolic blood pressure/diastolic blood pressure, pulse frequency, measurement time, and so on), and information measured by the scale/body composition meter 34 (body weight, body fat, skeletal muscle percentage, measurement time, and so on).

“System operational status data” includes user status data related to the operation of the system, such as periods in which the information terminals 21 through 23 are logged into the health management support system. The “external DB” includes the day's weather, temperature, and information of physical exam results for users (abdominal circumference, systolic blood pressure, diastolic blood pressure, neutral fat, fasting blood sugar values, and so on).

Meanwhile, although not shown in FIG. 3, survey response result information may also be stored in the data accumulation unit 2. “Survey response result information” refers to information from surveys related to a user's health management, collected for each user from a predetermined homepage provided by the server device 1.

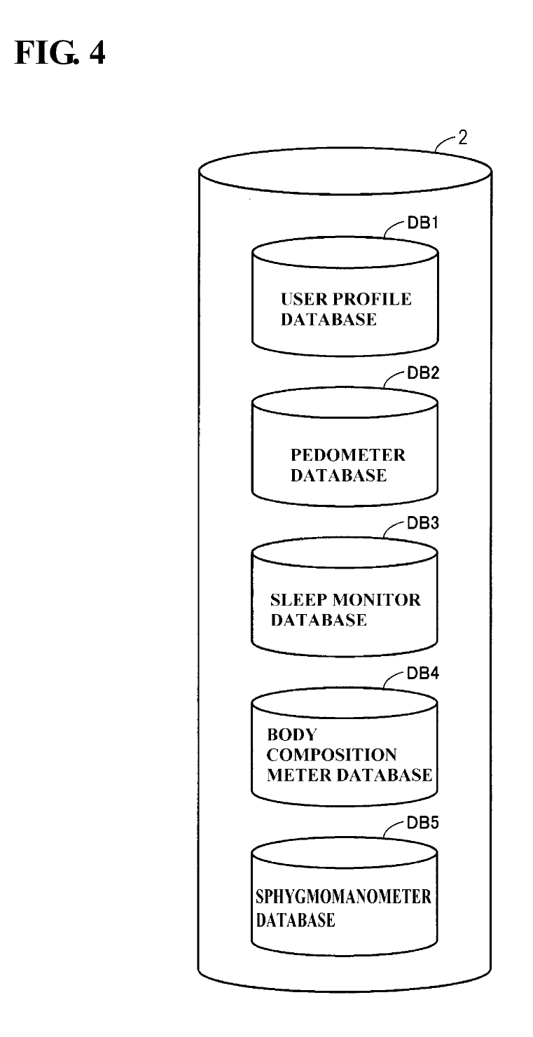

FIG. 4 illustrates types of databases stored in the data accumulation unit 2. For storing information received from users' healthcare devices 31 through 34 via the information terminals 21 through 23, the data accumulation unit 2 includes a user profile database DB1, a pedometer database DB2, a sleep monitor database DB3, a body composition meter database DB4, and a sphygmomanometer database DB5. Other types of databases may be stored in the data accumulation unit 2 as well. FIG. 4 shows an example of five databases, in order to simplify the descriptions.

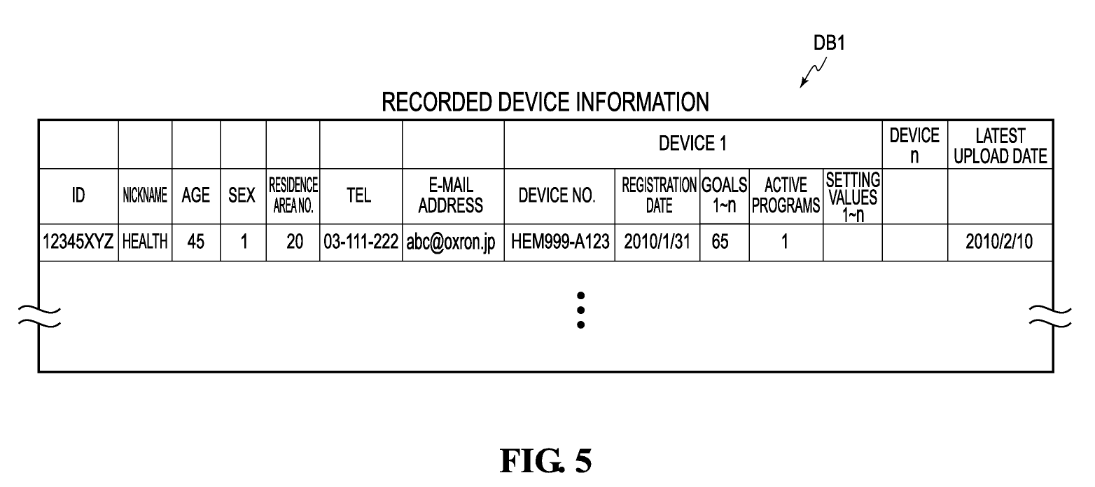

FIG. 5 illustrates an example of content in the user profile database DB1. For each user, information such as an ID (identifier) for uniquely identifying that user, a nickname, an age, a sex, an area of residence, a telephone number, and an email address is stored, along with information of a registered healthcare device, in the user profile database DB1. The information of the healthcare device includes, for each registered healthcare device, a date, a target value, information regarding a program being undertaken, device setting information (information downloaded to the device: height, sex, age, stride pitch, and so on), and other information (the most recent date and time on which data was uploaded, a login frequency, and so on). “Downloaded information” refers to information sent from the server device 1 to the respective information terminals.

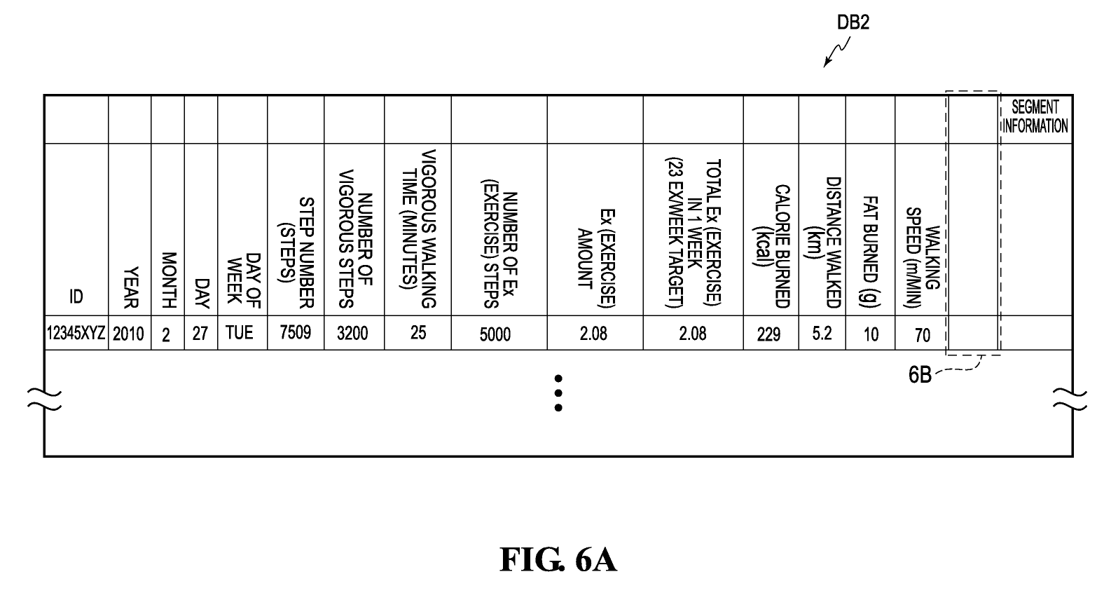

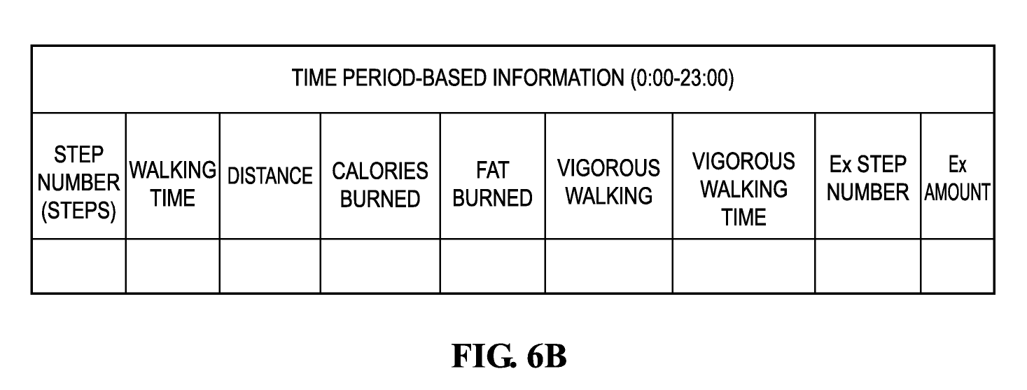

FIGS. 6 through 8 illustrate examples of content in the pedometer database DB2, the body composition meter database DB4, and the sphygmomanometer database DB5, respectively, shown in FIG. 4.

As shown in FIG. 6, uploaded information (measurement date, number of steps, time walked, distance walked, calories consumed, amount of fat burned, number of vigorous steps, time vigorously walked, number of exercise steps, exercise amount, time period-based information, segment information, and so on) and additional information (an ID for uniquely identifying the user, the day of the week of measurement, and so on) are stored in the pedometer database DB2 for each user. In FIG. 6, the time period-based information is shown at a higher level of detail. Note that the “uploaded information” refers to information sent from the information terminal to the server device 1.

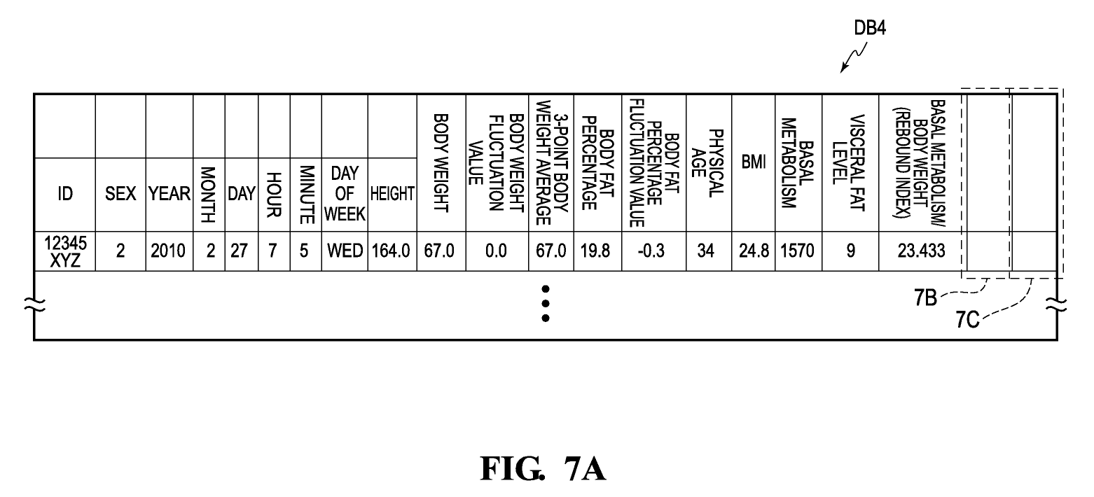

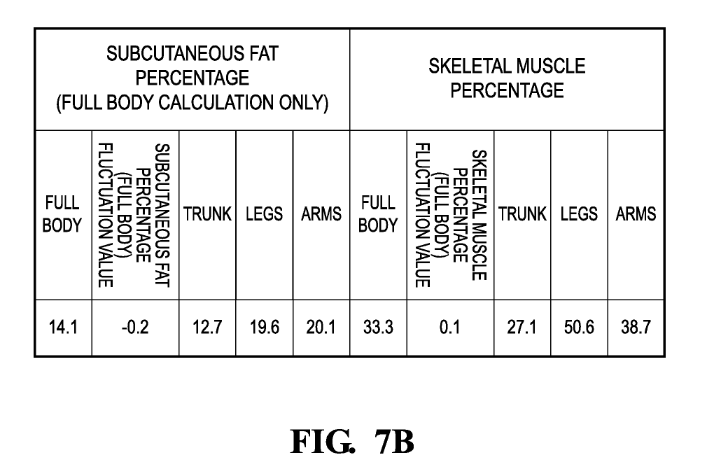

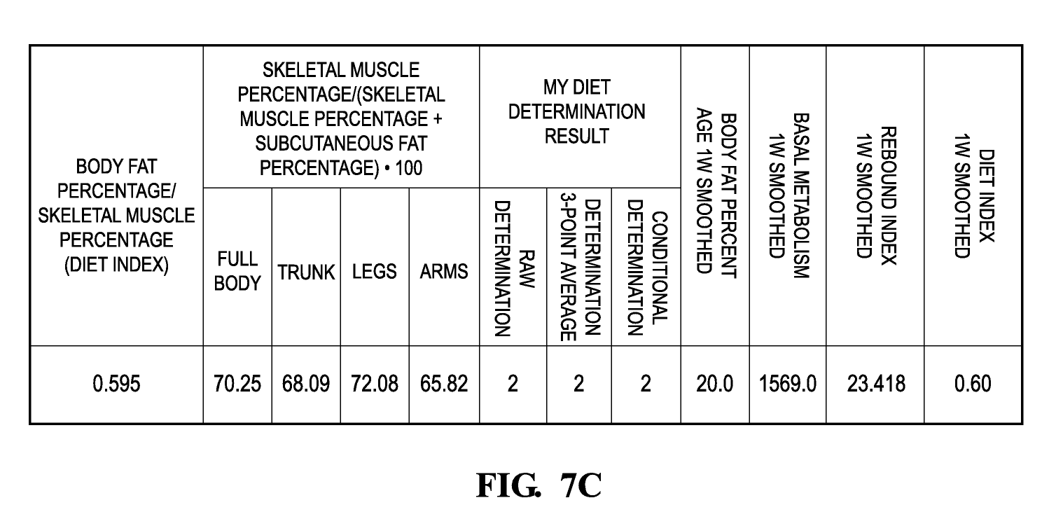

As shown in FIG. 7, uploaded information (sex, measurement date and time, body weight, body fat percentage, BMI (Body Mass Index), physical age, basal metabolism, skeletal muscle percentage, height, morning/evening execution results, and so on) and additional information (an ID for uniquely identifying the user, the day of the week of measurement, the value of fluctuation for one day, a rebound index, a diet index, personal diet determination results, and so on) are stored in the body composition meter database D134 for each user. In FIG. 7, some of the information is shown at a higher level of detail.

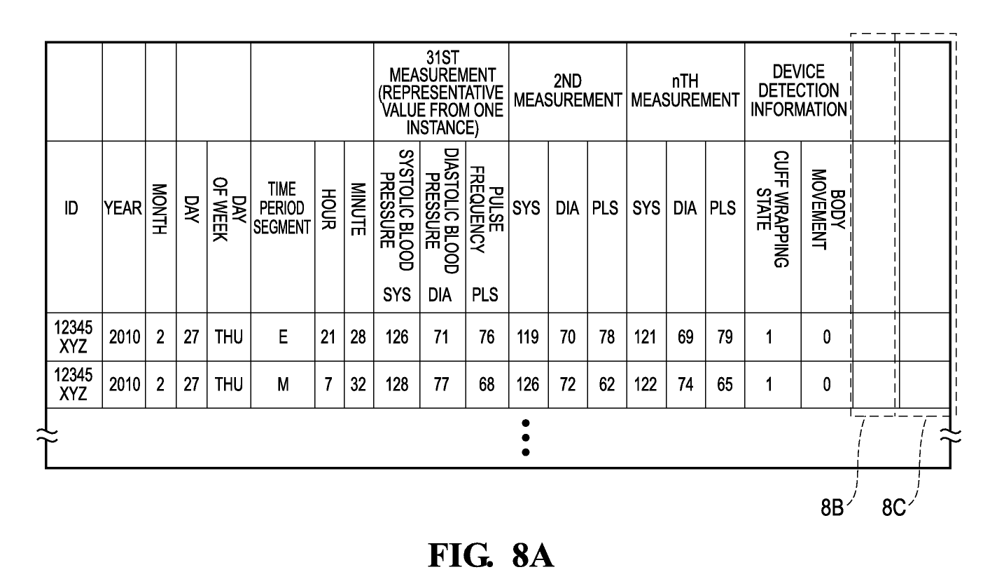

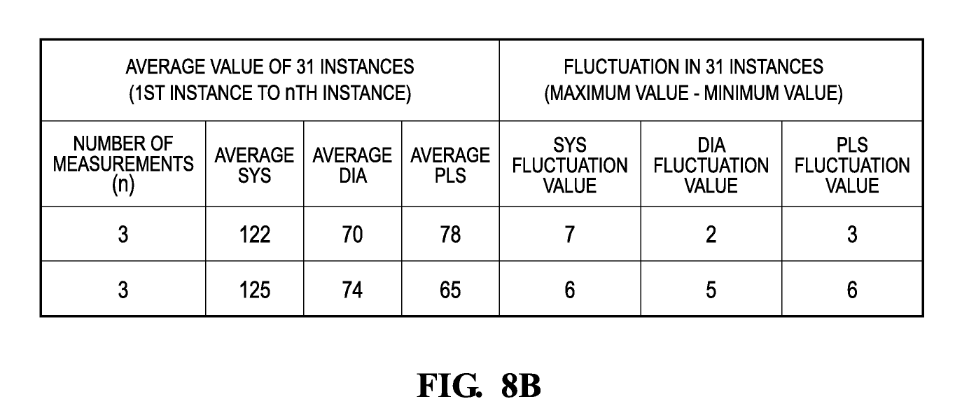

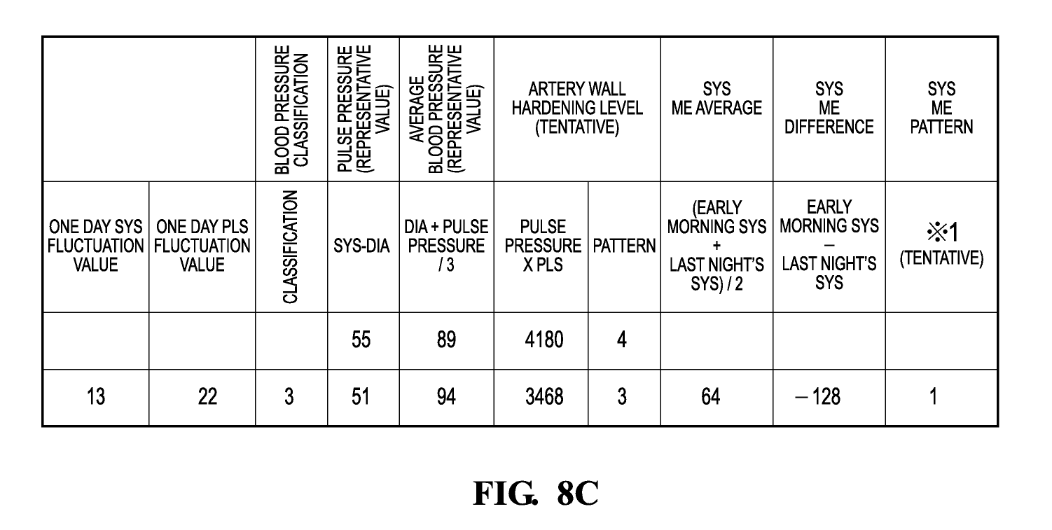

As shown in FIG. 8, uploaded information (measurement date and time, systolic blood pressure, diastolic blood pressure, pulse frequency, device detection information, and so on) and additional information (an ID for uniquely identifying the user, the day of the week of measurement, a pulse pressure, an average blood pressure, ME average, ME difference, the value of fluctuation for one day, and so on) are stored in the sphygmomanometer database DB5 for each user. Note that ME is an acronym for “morning” and “evening”. “ME average” refers to the average value for the systolic blood pressure after waking up (M) and before going to bed (E), whereas “ME difference” refers to the systolic blood pressure difference.

Uploaded information (measurement date, actual sleep time, time when the user fell asleep, time/length of time/number of times the user woke, snoring frequency, snoring level, and so on), additional information (an ID for uniquely identifying the user, the day of the week), and so on are stored in the sleep monitor database DB3 for each user. In FIG. 8, some of the information is shown at a higher level of detail.

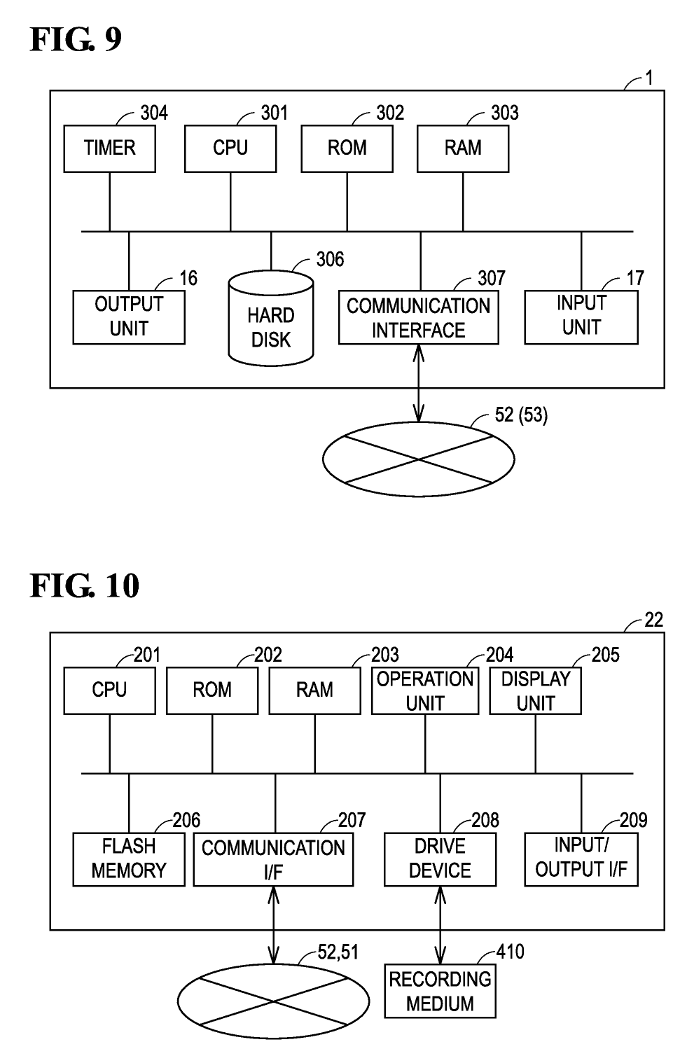

FIG. 9 illustrates the hardware configuration of the server device 1. The server device 1 includes: a CPU (Central Processing Unit) 301 for controlling the server device 1 as a whole; a ROM (Read-Only Memory) 302 that stores programs, data, and so on in advance; a RAM (Random Access Memory) 303 that stores various types of data; a timer 304; a hard disk 306; a communication I/F (interface) 307 for connecting the server device 1 to the communication path 52 (53); an output unit 16; and an input unit. The output unit 16 includes a display unit, a printing unit, an audio output unit, or the like. The input unit 17 includes a keyboard, a pointing device such as a mouse, or the like.

FIG. 10 illustrates the hardware configuration of the information terminals. Here, the information terminal 22 is shown as an example. As shown in FIG. 10, the information terminal 22 includes: a CPU 201 for controlling the information terminal 22 as a whole; a ROM 202 that stores programs, data, and so on in advance; a RAM 203 that records various types of data; an operation unit 204 for accepting instructions from a user, the input of various types of information, and so on; a display unit 205 for displaying information; a non-volatile memory, such as a flash memory, 206; a communication I/F 207 that is connected to the communication path 51 (52); a drive device 208 that writes and reads data to and from a recording medium 410; and an input/output I/F 209 for exchanging data with the healthcare devices 31 through 34.

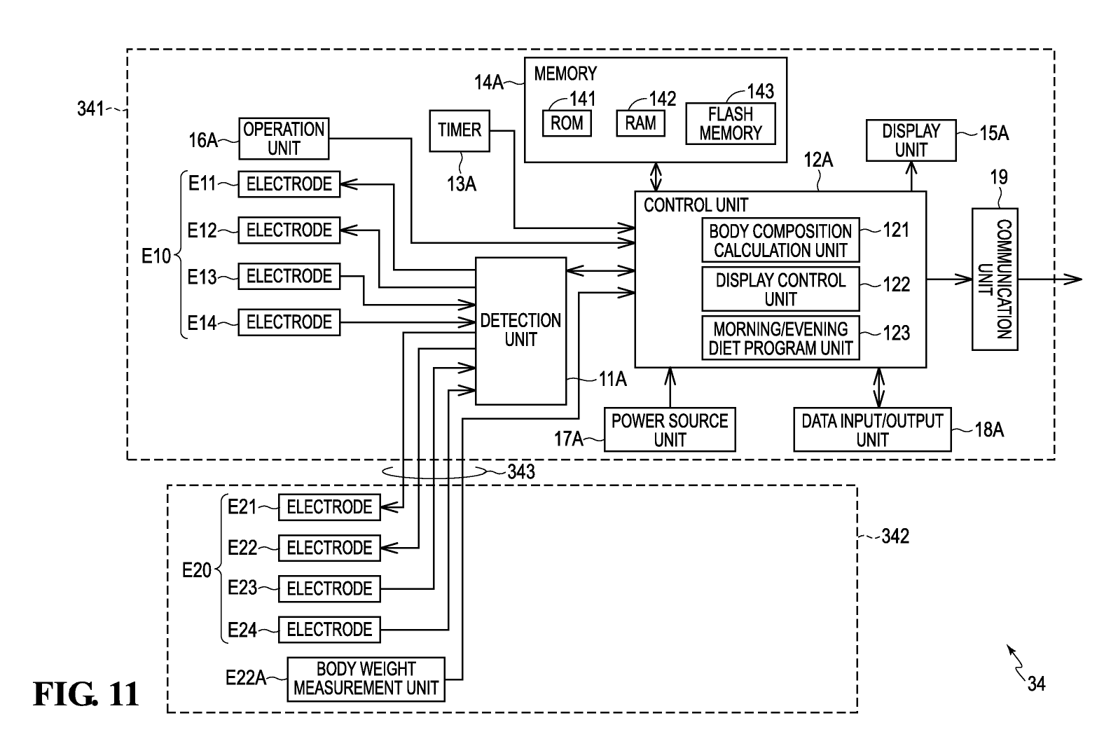

FIG. 11 is a block diagram illustrating the configuration of a healthcare device. Here, the scale/body composition meter 34 is illustrated as an example of a healthcare device. The scale/body composition meter 34 is configured in the same manner as proposed in JP 2007-296093A, filed by the present applicant, and therefore descriptions thereof will be simplified here.

The scale/body composition meter 34 includes a body weight measurement function and a function for measuring the body composition of a user by measuring an impedance. With respect to the impedance, impedances are measured for different areas of a measurement subject using multiple electrodes E11 through E14 and E21 through E24, which are caused to come into contact with multiple predetermined corresponding areas of the user's body. The scale/body composition meter 34 includes: an upper limb unit 341 that a user can grip with both hands; a lower limb unit 342 on which the user can place both feet; and a cable 343 that electrically connects the upper limb unit 341 and the lower limb unit 342.

In addition to hand electrodes E10, a display unit 15A, and an operation unit 16A, the upper limb unit 341 further includes: a detection unit 11A for detecting a potential difference between at least the hands and feet (that is, for the whole body) of the user when a current is applied between the hands and feet by both the hand electrodes E10 and foot electrodes E20; a control unit 12A for controlling the scale/body composition meter 34 as a whole; a timer 13A for measuring a date and time; a memory 14A for storing various types of data and programs; a power source unit 17A for supplying power to the control unit 12A; a communication unit 19 for exchanging data with the information terminals 21 through 23; and a data input/output unit 18A for making inputs/outputs to and from an external device.

In addition to the foot electrodes E20, the lower limb unit 342 includes a body weight measurement unit 22A for measuring the user's body weight. The body weight measurement unit 22A is configured of, for example, a sensor.

The memory 14A includes a ROM 141 that stores programs, data, and so on in advance, a RAM 142 that records various types of data, and a non-volatile memory, such as a flash memory, 143. An example of the content of the flash memory 143 will be given later.

The display unit 15A is configured of, for example, an LCD (liquid-crystal display).

The operation unit 16A includes, for example, multiple buttons. The operation unit 16A may include, for example, a power button for instructing the power to be turned on/off; a memory button for instructing past measurement information to be displayed; a measure button for instructing the start of measurement; and multiple, such as four, personal number buttons that enable multiple users to use the scale/body composition meter 34. In the present embodiment, descriptions will be given assuming that the operation unit 16A includes four personal number buttons in this manner.

The detection unit 11A switches electrodes under the control of the control unit 12A. The detection unit 11A furthermore applies a current between both hands or both feet of the user through either the hand electrodes E10 or the foot electrodes E20, and detects a potential difference between both hands or both feet. Information of the detected potential difference is outputted to the control unit 12A.

The control unit 12A is configured of, for example, a CPU. The control unit 12A includes: a body composition calculation unit 121 for calculating two or more types of body compositions for a user based on programs stored in the ROM 141 in advance; a display control unit 122 for controlling the display of the results of the calculations performed by the body composition calculation unit 121 in the display unit 15A based on a specification program, which will be described in detail later; and a morning/evening diet program unit 123 for controlling a morning/evening diet program function, which will be described later.

The body composition calculation unit 121 measures a full-body impedance, an inter-hand impedance, and an inter-foot impedance, based on potential differences between the hands and feet, between both hands, and between both feet, as detected by the detection unit 11A. The body composition calculation unit 121 then calculates various types of body compositions of the user based on the measured impedances.

In the present embodiment, the body composition calculation unit 121 calculates four types of body compositions, such as a body fat percentage, a skeletal muscle percentage, a visceral fat surface area (also called a “visceral fat level”), and a basal metabolism, based on the full-body impedance, the inter-hand impedance, and the inter-foot impedance. The body compositions that are calculated are not limited thereto, however.

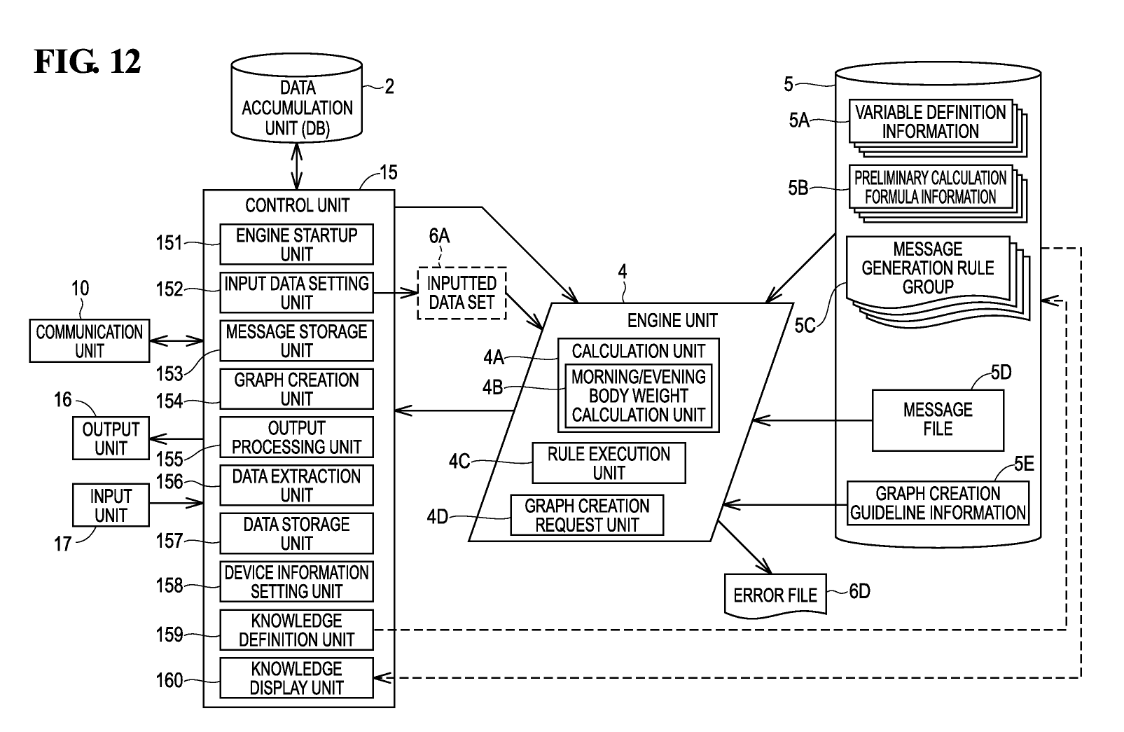

FIG. 12 illustrates a functional configuration in the server device 1 for analyzing physical information of a user and generating a message based on the results of that analysis. As shown in FIG. 12, the server device 1 includes the engine unit 4 for performing analysis and generating messages, and a control unit 15 for controlling the engine unit 4. The data in the knowledge file group 5 is referred to by the engine unit 4, and error data produced as a result of the analysis performed by the engine unit 4 is stored in an error file 6D.

The knowledge file group 5 includes: preliminary calculation formula information 5B; variable definition information 5A such as variables in which are set data from the results of calculations based on the preliminary calculation formula information 5B; a message generation rule group 5C specifying rules (command code) for generating the message 7 through a program written in a predetermined interpreter language; a message file 5D; and graph creation guideline information 5E.

The variable definition information 5A, the preliminary calculation formula information 5B, and the message generation rule group 5C each include information/rules referred to by the engine unit 4 when carrying out a message generation operation at an immediate execution timing, information/rules referred to by the engine unit 4 when carrying out a message generation operation on a weekly basis, and information/rules referred to by the engine unit 4 when carrying out a message generation operation on a monthly basis.

The message file 5D holds, in advance, multiple types of messages 7 and identification values uniquely identifying those messages 7 in association with the messages 7. The graph creation guideline information 5E holds, in advance, multiple types of graph creation guidelines indicating procedures (command code) for creating the graph 8, and identification values uniquely identifying those graph creation guidelines in association with the guidelines.

In the present embodiment, the various elements of the engine unit 4 can analyze the information collected from the users' healthcare devices 31 through 34 and execute operations for generating a message based on the results of the analysis immediately (that is, upon the data being collected), on a weekly basis (each week from weeks one to four), and a monthly basis, for the information from each user.

Based on a request from the control unit 15, the engine unit 4 switches, for the variable definition information 5A, the preliminary calculation formula information 5B, and the message generation rule group 5C, to referring to the variable definition information 5A, the preliminary calculation formula information 5B, and the message generation rule group 5C that correspond to the stated request.

The engine unit 4 includes: a calculation unit 4A having a function for calculating characteristic values (including regression coefficients, Max, Min, average values, standard deviations, mode values, attributes, and so on) based on measured data by carrying out computational processes on the various types of measured data from the physical information collected from the users, based on predetermined calculation formulas read out from the preliminary calculation formula information 5B (functions, four arithmetic operations, Boolean operations, comparison operations, and so on); a rule execution unit 4C that analyzes rules in the message generation rule group 5C based on the results of the calculations and outputs the results of the analysis; and a graph creation request unit 4D that refers to the graph creation guideline information 5E based on the results of the analysis and outputs a graph creation request based on the results of the reference.

The rule execution unit 4C includes an interpreter. The interpreter interprets and executes program command code of the message generation rule group 5C. The engine unit 4 searches the message file 5D based on the result of the execution (values), reads out the message 7 associated with an identification value that matches the stated result of the execution, and outputs that message 7 to the control unit 15. Meanwhile, the result of the execution performed by the rule execution unit 4C is outputted to the graph creation request unit 4D. The graph creation request unit 4D searches the graph creation guideline information 5E based on the result of the execution performed by the rule execution unit 4C (a value), reads out the graph creation guideline associated with an identification value that matches the stated result of the execution, and outputs that graph creation guideline along with the graph creation request to the control unit 15.

Although a processing system that employs an interpreter for analysis and message generation is applied in the present embodiment, it should be noted that the processing system applied is not limited to an interpreter, and may be another processing system instead.

In this manner, by generating and presenting the message 7 and the graph 8 corresponding to the results of an analysis that can be carried out by the rule execution unit 4C on lifestyle patterns/states of health based on physical information measured from a user, it is possible to provide, to the user, advice for improving his/her lifestyle patterns in order to enable the user to achieve his/her goals.

The calculation unit 4A includes a morning/evening body weight calculation unit 4B for executing a morning/evening diet program, described later.

The control unit 15 includes: an engine startup unit 151 for starting up the engine unit 4; an input data setting unit 152 that takes data read out from the data accumulation unit 2 as its input, edits data into an inputted data set 6A, and outputs the inputted data set 6A to the engine unit 4; a message storage unit 153 that stores the message 7 based on data provided via the communication unit 10 or the input unit 17; a graph creation unit 154 (this corresponds to the graph creation unit 6 shown in FIG. 2); an output processing unit 155; a data extraction unit 156 (this corresponds to the data extraction unit 3 shown in FIG. 2) that searches the data accumulation unit 2 and outputs data based on the result of the search; a data storage unit 157 (this corresponds to the data storage unit 12 shown in FIG. 2) for storing data provided by the communication unit 10 or the input unit 17 in the data accumulation unit 2; a device information setting unit 158 (this corresponds to the device information setting unit 11 shown in FIG. 2); a knowledge definition unit 159 (this corresponds to the knowledge definition unit 13 shown in FIG. 2); and a knowledge display unit 160 (this corresponds to the knowledge display unit 14 shown in FIG. 2).

The engine startup unit 151 starts up the engine unit 4 based on information inputted from the communication unit 10 or the input unit 17. The message storage unit 153 temporarily stores the message 7 outputted from the engine unit 4 in a predetermined storage region. The graph creation unit 154 creates graph data in response to the graph creation request outputted from the engine unit 4. Specifically, the data extraction unit 156 searches the data accumulation unit 2 based on the graph creation guidelines, reads out data, and outputs the data to the graph creation unit 154. The graph creation unit 154 edits the data read out from the data accumulation unit 2 into the graph 8 based on the graph creation guidelines, and outputs the graph 8.

The device information setting unit 158 outputs, to the communication unit 10, destination information of the data sent from the communication unit 10. As the destination information, the device information setting unit 158 outputs an email address read out from the user profile database DB1 based on a user ID.

The output processing unit 155 outputs various types of data, such as the message 7, the graph 8, and so on, via the output unit 16. The knowledge definition unit 159 updates the information within the knowledge file group 5 based on information inputted from the input unit 17. Through this, the information in the message file 5D and the graph creation guideline information 5E can be updated (added/changed/deleted) independent from the engine unit 4.

The knowledge display unit 160 outputs the information within the knowledge file group 5 via the output unit 16. Through this, the information in the message file 5D and the graph creation guideline information 5E can be updated while confirming the information via the output unit 16. The content of the error file 6D can also be outputted by the output processing unit 155 via the output unit 16.

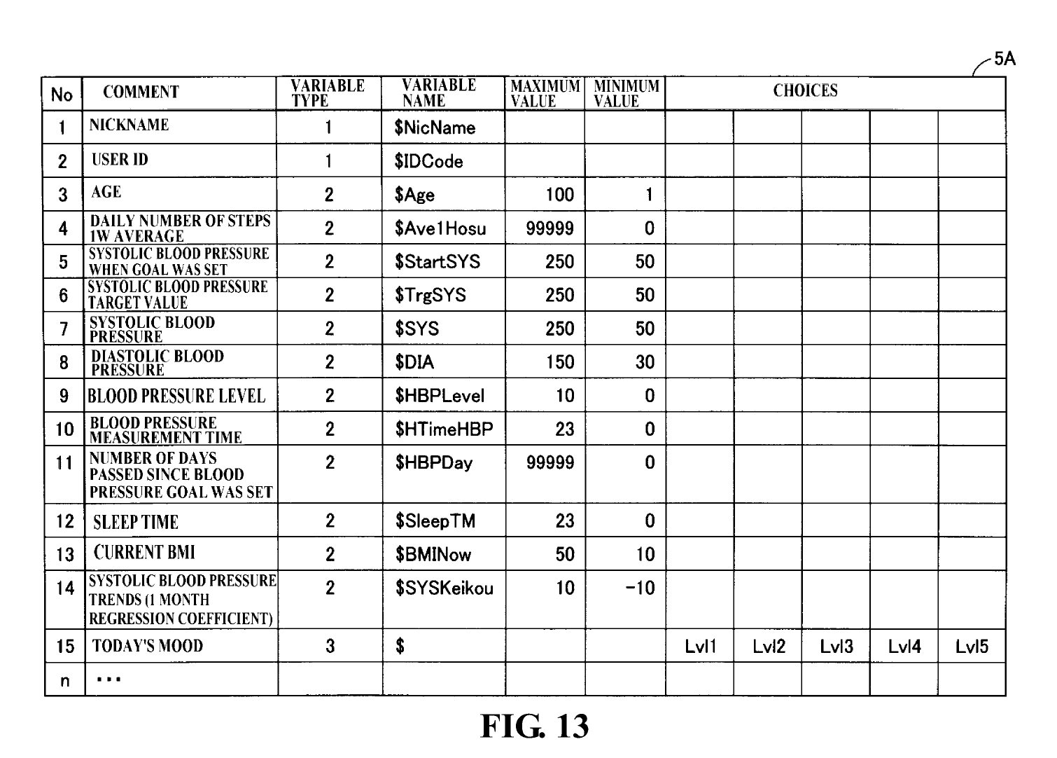

FIG. 13 illustrates an example of variables defined by the variable definition information 5A. The variables in the variable definition information 5A are configured of system variables (variables in which the profile, information for data processing, operation information, information of the collected healthcare data, and so on are set) and internal variables (variables in which calculation results outputted from the preliminary calculation formula information 5B are set). Here, a variable indicates a single type of storage region, and information (a result) being set in a variable indicates that the information (the result) is written (stored) in that storage region. The variable names in FIG. 13 indirectly indicate the addresses of those storage regions. Accordingly, the respective elements of the engine unit 4 can input/output data required for processing via the variables defined by the variable definition information 5A. “Storage region” refers to, for example, a region in the RAM 303.

Written in the preliminary calculation formula information 5B are calculation formulas referred to in the case where calculations are necessary, such as additions carried out in advance based on the values in the inputted data set 6A. The types of calculations include functions (regression coefficients for a certain period, Max, Min, average values, standard deviations, mode values, attributes, calculations for degrees of change, and so on), four arithmetic operations, Boolean operations, comparisons, and so on. The calculation unit 4A executes computations in accordance with the calculation functions in order to execute message generation rules.

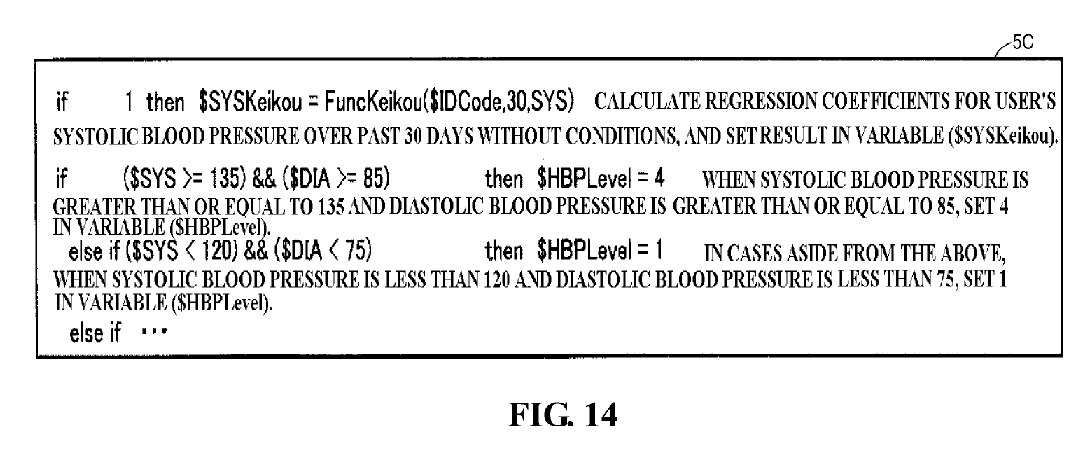

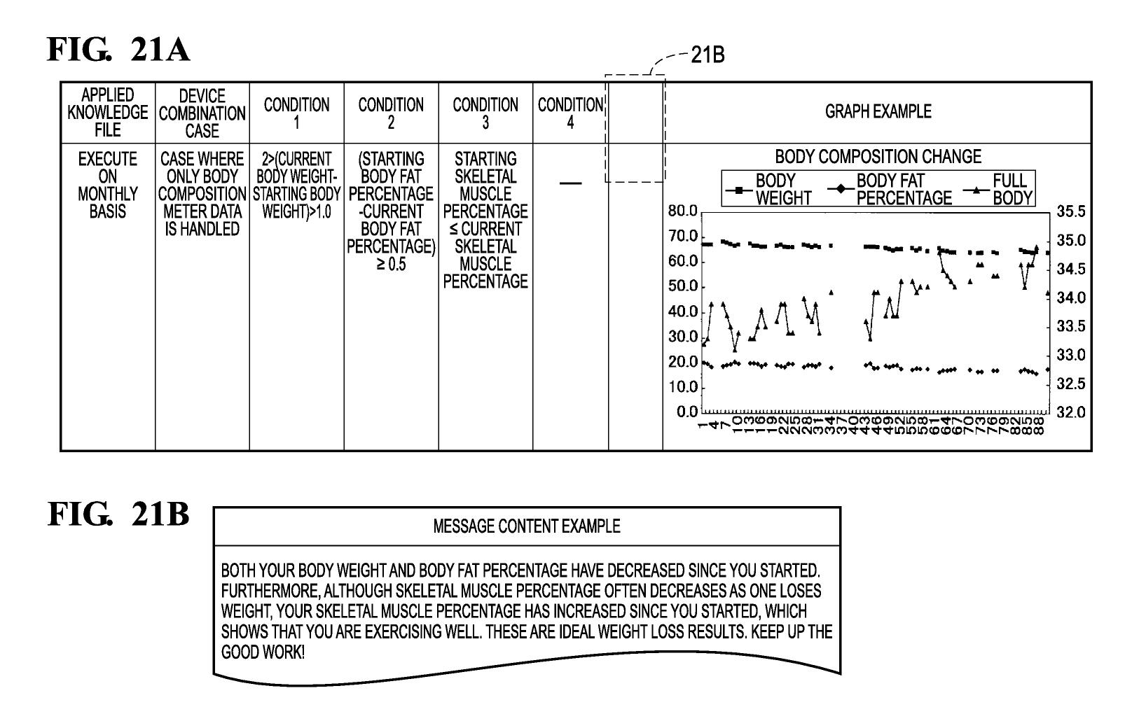

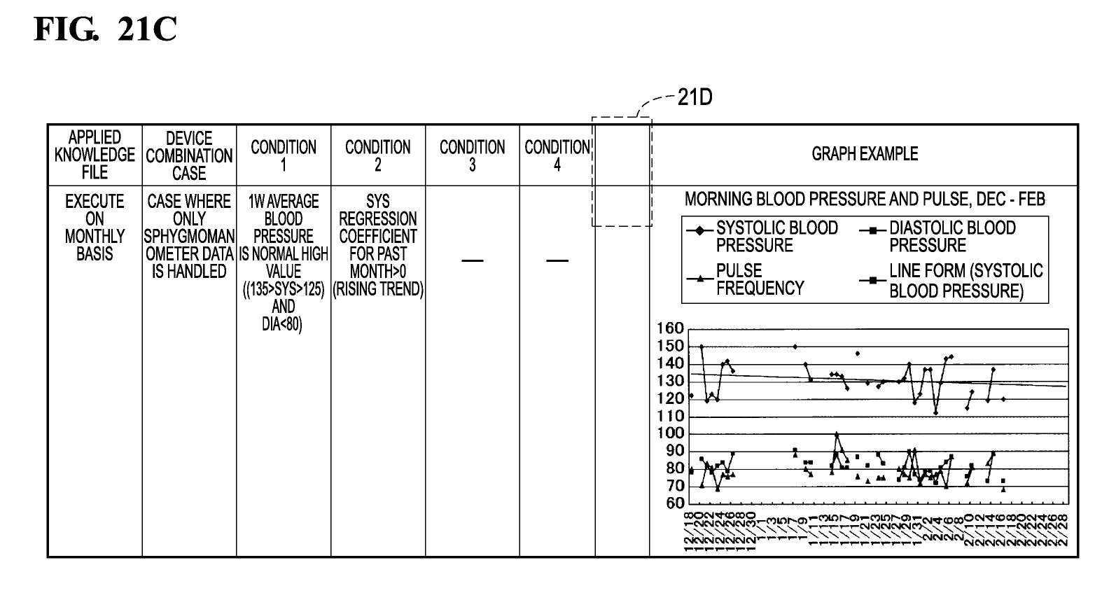

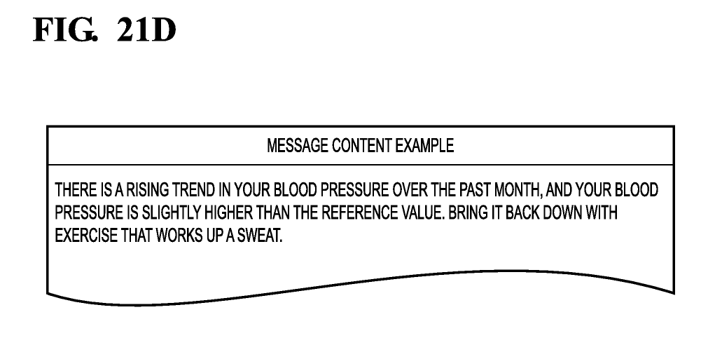

An example of the message generation rule group 5C that incorporates the preliminary calculation formula information 5B will be described next with reference to FIG. 14. As shown in FIG. 14, the rule for message generation is written as conditional branches, or if (condition) then (condition) else (condition) if, and so on. In the conditional branches, conditions (conditional expressions) are written using the various types of variables indicated in FIG. 13. These conditions indicate, for example, “condition 1” through “condition 4” as shown in FIGS. 21 through 23, described later. The formulas in the preliminary calculation formula information 5B are applied in the calculation formulas or the items in the calculation formulas written in each condition.

The rule execution unit 4C sequentially executes the rules while setting the variable values from the inputted data set 6A in the variables for each condition in the message generation rule group 5C, and outputs execution results (values) specifying output text (the message 7) and guidelines for the graph creation guideline information 5E that conforms to the conditions. Calculation formulas for detecting the presence/absence of relationships between two or more types of physical information and the degree of correlation therebetween, as well as formulas for comparisons with predetermined reference values, can be expressed in the conditional expressions of the rules; accordingly, by executing the rules, evaluation values based on the mutual relationships between the pieces of physical information and the results of comparisons with the predetermined reference values can be detected.

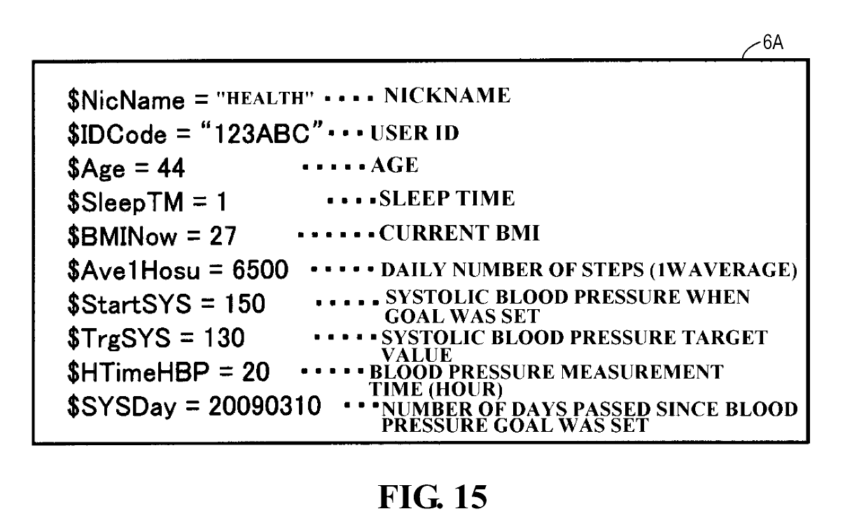

FIG. 15 illustrates an example of the inputted data set 6A. The input data setting unit 152 sets the values in the information read out from the data accumulation unit 2 in the respective corresponding variables read out from the variable definition information 5A. The inputted data set 6A in FIG. 15 shows a state in which values (data) are set in correspondence with the respective variables.

Body Weight/Body Composition Measurement Process

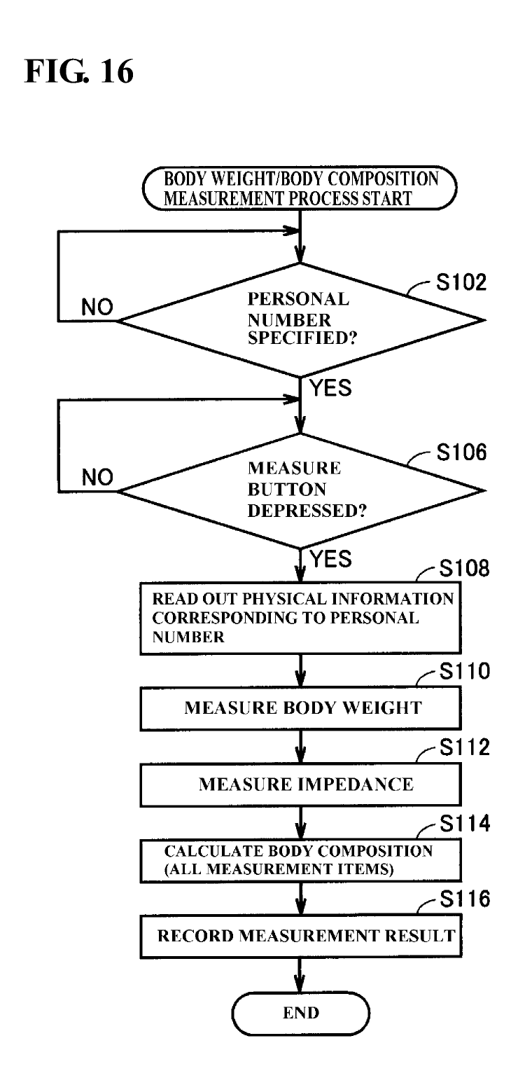

A measurement process executed by the scale/body composition meter 34 will now be described with reference to FIG. 16.

First, the control unit 12A determines whether or not a personal number has been specified by the user (step S102). In other words, it is determined whether or not one of the four buttons has been depressed by the user. The control unit 12A stands by until a personal number has been specified (NO in step S102). In the case where it has been determined that a personal number has been specified (YES in step S102), the process advances to step S106.

In step S106, the control unit 12A determines whether or not the measure button has been depressed, and stands by until the measure button is depressed (NO in step S106). When the measure button is depressed (YES in step S106), the process advances to step S108.

In step S108, the body composition calculation unit 121 reads out physical information (height, age, sex) corresponding to the personal number specified by the user from the flash memory 143 in which that information is stored in advance. The physical information that has been read out is recorded in an internal memory.

Next, the body composition calculation unit 121 measures a body weight based on a signal from the body weight measurement unit 22A (step S110). The measured body weight value is temporarily recorded in the flash memory 143.

Next, the body composition calculation unit 121 executes an impedance measurement process (step S112). The respective impedance values that have been measured are recorded in the internal memory.

The body composition calculation unit 121 calculates four types of body compositions of the user based on the respective pieces of data temporarily recorded in the internal memory and predetermined calculation formulas and the like (step S114). Note that here, body compositions corresponding to all four types of measurement items are calculated. Then, the control unit 12A records the measurement results, or in other words, the values of the body compositions calculated in step S114, in the internal memory (step S116). The results of measuring the body weight and the body compositions are then displayed. The measurement process then ends.

System Process

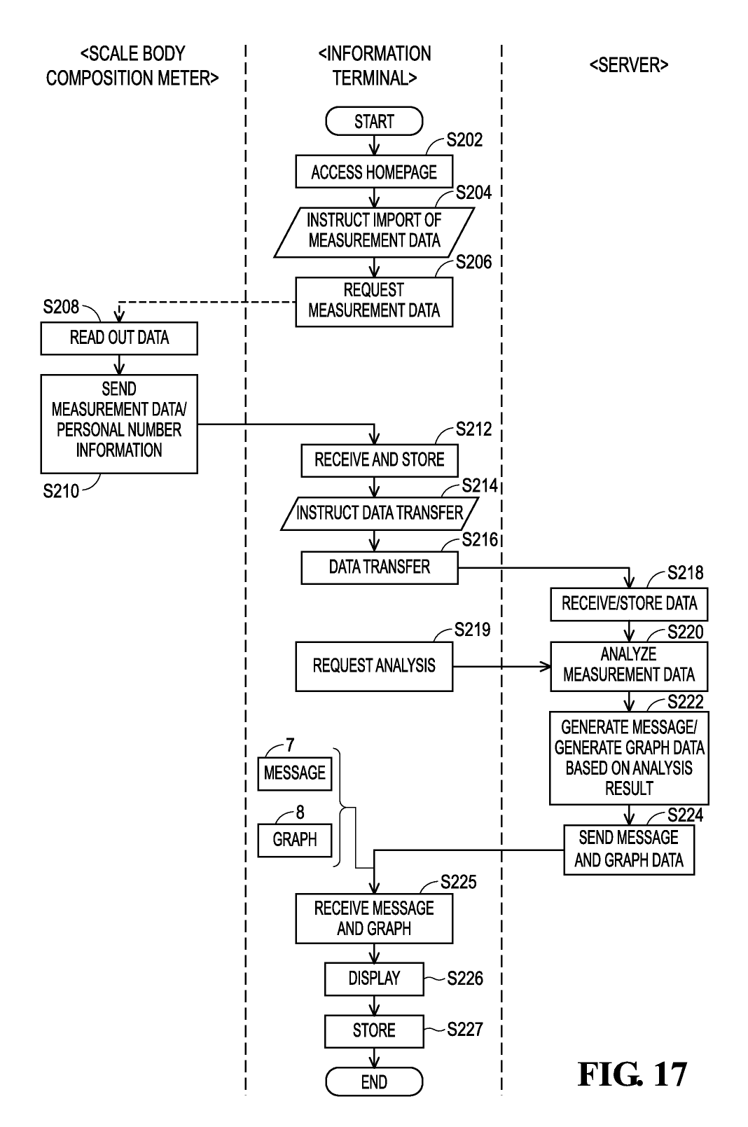

FIG. 17 is a flowchart illustrating operations performed by the health management support system according to an embodiment of the present invention. FIG. 17 illustrates a flow in which data is sent to the server device 1 from the scale/body composition meter 34 via the information terminal 22, and a flow in which data analysis is carried out having set the timing of the execution of the various elements in the engine unit 4 to “monthly basis”.

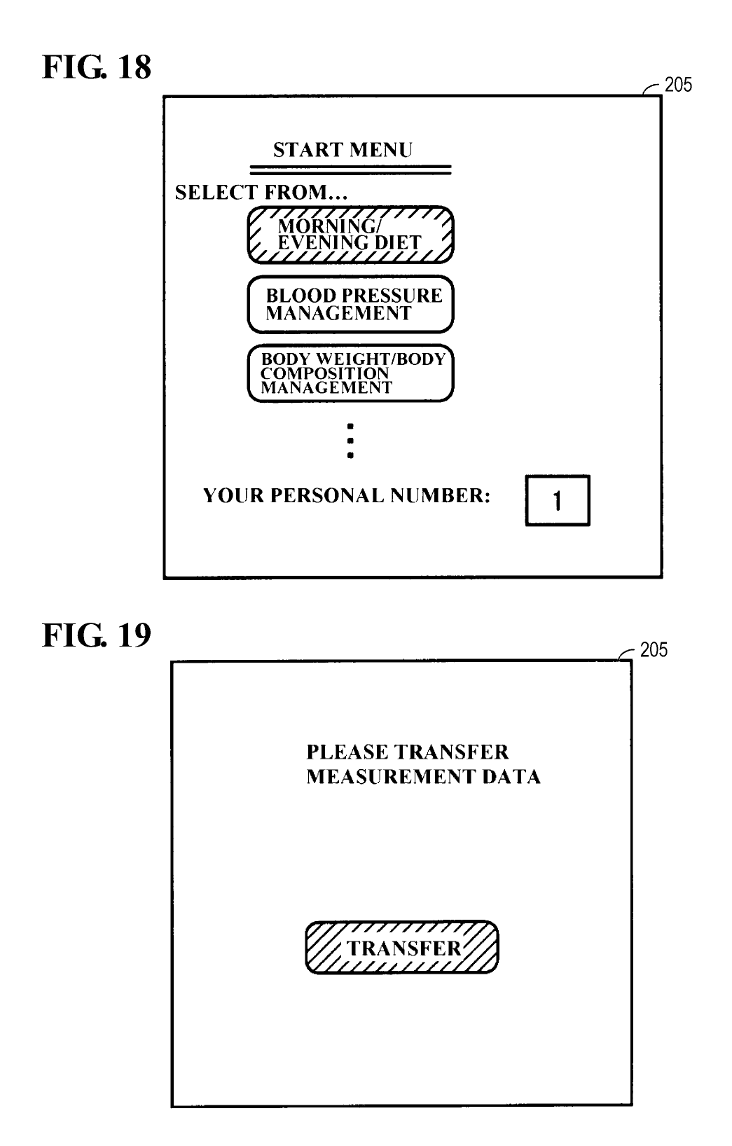

First, the flow for sending data will be described. As shown in FIG. 17, the information terminal 22 accesses a homepage provided by the server device 1 based on an instruction from the user (step S202). At this time, the transmission terminal 200 displays, in the display unit 205, a menu screen for the health management support system that has been sent from the server device 1. FIG. 18 illustrates an example of the screen that is displayed.

As shown in FIG. 18, the menu screen used when selecting a program in the health management support system displays items (buttons) indicating the respective programs along with an input field for inputting the user's personal number.

When such a screen is displayed in the display unit 205, the user selects a program and inputs his or her personal number. In FIG. 18, “body weight/body composition management” is selected as the program, and 1 is inputted as the personal number. The data of the inputted personal number is temporarily recorded in the RAM 203.

After this, when an instruction to import measurement data is inputted by the user (step S204), the information terminal 22 prompts the user to send the measurement data (step S206). Specifically, for example, a message reading “please send body weight/body composition measurement data” is displayed in the display unit 205.

Meanwhile, in the scale/body composition meter 34, the body weight/body composition measurement data is read out from the flash memory 143 as a result of the user operating the operation unit 16A (step S208), and a process for sending that data to the information terminal 22 via the communication unit 19 is executed. The scale/body composition meter 34 outputs the physical information and the measurement data of the user to the information terminal 22 (step S210). Specifically, in step S208, the control unit 12A of the scale/body composition meter 34 reads out the personal number inputted by the user, age data, sex data, and height data stored in correspondence therewith, and the most recent measurement data of the user stored in the flash memory 143 (weight, body fat percentage, skeletal muscle percentage, visceral fat level, basal metabolism, and so on), and sends the read-out data to the information terminal 22 via the communication unit 19.

The information terminal 22 receives the physical information and the measurement data through the input/output I/F 209, and temporarily stores that information and data in the flash memory 206 (step S212). Upon doing so, a screen such as that shown in, for example, FIG. 19 is displayed in the display unit 205. As shown in FIG. 19, a message reading “please transfer measurement data” and a button for instructing the transfer are displayed in the display unit 205.

When the user operates the operation unit 204 and makes an input instructing the transfer of the measurement data while such a screen is being displayed (step S214), the information terminal 22 transfers the physical information and measurement data received in step S212 to the server device 1 (step S216). The personal number information received in step S212 is temporarily recorded in the RAM 203.

Although the transfer of data from the information terminal 22 to the server device 1 is described as being executed in response to an instruction from the user, it should be noted that the transfer method is not limited thereto. For example, the information terminal 22 may automatically transfer the measurement data to the server device 1 as soon as the measurement data has been successfully received from the scale/body composition meter 34.

The server device 1 receives the physical information and measurement data from the information terminal 22, and stores that information and data in the body composition meter database DB4 of the data accumulation unit 2 as uploaded information (step S218). Through this, the server device 1 can collect information from the scale/body composition meter 34.

Next, a flow of analysis performed using the engine unit 4 will be described. The user operates operation unit 204 at the information terminal 22, and inputs his or her user ID along with a request for “monthly analysis of body weight/body composition data”. The inputted request is sent to the server device 1 (step S219). The “user ID” referred to here corresponds to the personal number.

The request for analysis may also correspond to a data input made by the user. In addition, the date and time of analysis request may be automatically recognized based on the number of days that have passed since a day the user requested messages to start, a day set as a target, or the like.

Upon receiving the analysis request, the CPU 301 of the server device 1 reads out, in response to the request, the user's measurement data for the past month from the body composition meter database DB4 in the data accumulation unit 2, based on the request and the received ID. The read-out measurement data is then analyzed by the engine unit 4 (step S220). The message 7 and the graph 8 are then generated based on the result of the analysis (step S222). Detailed descriptions of steps S220 and S222 will be given later.

The destination information outputted from the device information setting unit 11 is added to the data generated in step S222 by the communication unit 10, and the data is then sent to the information terminal 22 (step S224).

The information terminal 22 receives the information of the message 7 and the graph 8 sent by the server device 1 (step S225), and displays that information in the display unit 205 (step S226). An example of this display will be described later.

The data of the received message 7 and graph 8 are stored in the RAM 203 on a user-by-user basis (step S227). After this, the process ends.

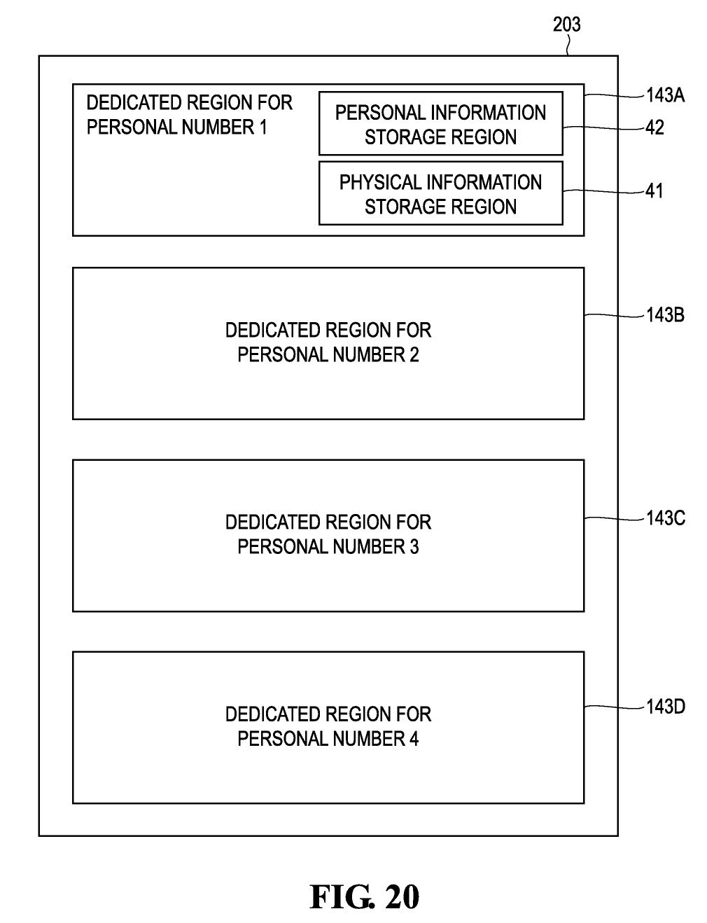

FIG. 20 illustrates an example of content stored in the RAM 203 of the information terminal 22. As shown in FIG. 20, the AM 203 includes regions 143A through 143D for storing information related to users in correspondence with those users' personal numbers. Each of the regions 143A through 143D includes a personal information (that is, the information stored in the user profile database DB1 shown in FIG. 5) storage region 42 and a physical information storage region 41 for storing physical information, for the user corresponding to the personal number in question. Data regarding health management received from the server device 1 (that is, data of the message 7 and the graph 8) is stored in the physical information storage region 41. It is assumed that the regions 14313 through 143D corresponding to other personal numbers include the same types of storage regions as those in the region 143A. Here, it is assumed that the content of the storage regions 42 is stored in the user profile database DB1 in advance on an ID-by-ID basis.

Next, the specific processes carried out in the stated steps S220 and S222 will be described.

Specific Examples of Measurement Data Analysis Process/Message and Graph Generation Process

In the present embodiment, as a process for analyzing the measurement data, advice (the message 7, the graph 8) that is to be provided to the user for health management is generated based on one or more types, and preferably, on multiple types of physical information collected from the user.

In the processes of steps S220 and S222, the control unit 15 outputs, to the engine unit 4, the user ID inputted via the communication unit 10 and the request for “monthly analysis of body weight/body composition data” (called simply a “request” hereinafter), and the engine startup unit 151 starts up the engine unit 4.

The data extraction unit 156 of the control unit 15 searches the body composition meter database DB4 in the data accumulation unit 2 based on the user ID and the request, reads out that user's measurement data for the past month based on time measurement data measured by the timer 304, and outputs the measurement data to the input data setting unit 152.

The input data setting unit 152 generates the inputted data set 6A by setting the data inputted from the data extraction unit 156 in the respective variables of the monthly body weight/body composition variable definition information 5A, and outputs the generated inputted data set 6A.

The calculation unit 4A of the engine unit 4 reads out the monthly body weight/body composition preliminary calculation formula information 5B, substitutes the variables in the respective calculation formulas that have been read out with the values of the corresponding variables in the inputted data set 6A, and executes computations in accordance with the calculation formulas. The results of the calculations are outputted to the rule execution unit 4C.

The rule execution unit 4C substitutes the variables in the inputted data set 6A and the calculation result values for the conditions of the respective rules in the monthly body weight/body composition message generation rule group 5C, and executes the conditions in sequence. The results of the execution are outputted to the graph creation request unit 4D. Based on the results of the executions performed by the rule execution unit 4C, the engine unit 4 reads out the message 7 associated with those execution results from the message file, and outputs the read-out message 7 to the control unit 15.

Meanwhile, based on the results of the executions performed by the rule execution unit 4C, the graph creation request unit 4D reads out the graph creation guideline associated with an identification value that matches the stated execution results from the graph creation guideline information 5E, and outputs that graph creation guideline along with the graph creation request to the control unit 15.

When the graph creation request is inputted, the graph creation unit 154 of the control unit 15 generates the graph 8 based on the graph creation guideline using the data of the stated user read out from the data accumulation unit 2, and outputs the generated graph 8.

The communication unit 10 adds, to the message 7 and the graph 8 based on the analysis result, the destination information (that is, the email address searched out and read out from the user profile database DB1 by the device information setting unit 158 based on the user ID), and outputs the resulting data to the communication path 52.

The information terminal 22 displays the message 7 and graph 8 received from the server device 1.

Through this, the analysis of one month's worth of data measured by the scale/body composition meter 34, and health management advice (the message 7, the graph 8) based on the result of that analysis, are provided to the user.

Although the aforementioned physical information analysis uses two types of information, or body weight and body composition, the number and types of physical information that are combined are not limited thereto; blood pressure and body composition, blood pressure, body weight, and body composition, and so on may be combined as well.

Analysis Examples and Display Examples

Examples of the details of analyses performed by the server device 1 on a user's physical information according to the present embodiment will be described hereinafter.

First, FIGS. 21A and 21B illustrate two cases in which analysis is executed on a “monthly basis”. The former illustrates an example in which two types of physical information, or body weight and body composition, measured by the scale/body composition meter 34 have been analyzed, whereas the latter illustrates an example in which blood pressure information measured by the sphygmomanometer 32 has been analyzed. In FIGS. 21A and 21B, examples of the content of the messages are shown in greater detail.

In FIGS. 21A and 21B, the types of conditions (condition 1 through condition 4) indicated by the rules in the message generation rule group 5C and executed by the rule execution unit 4C are shown for each case, and examples of the content of the message 7 and the graph 8 outputted as a result of the analysis are also shown. The message 7 introduces methods for measuring with or using the healthcare device, how to read the data displayed, and so on; this includes changes in the measurement data, introductions of knowledge and evidence, encouragement, points of caution, meals and exercise for achieving goals, and the like.

With the case shown in FIG. 21A, the graph 8 is a polygonal line graph showing, as time passes, the changes in analysis results based on two types of physical information obtained from the data measured by the scale/body composition meter 34, or body weight and body fat; the message 7, based on the analysis results for both pieces of physical information, is also displayed.

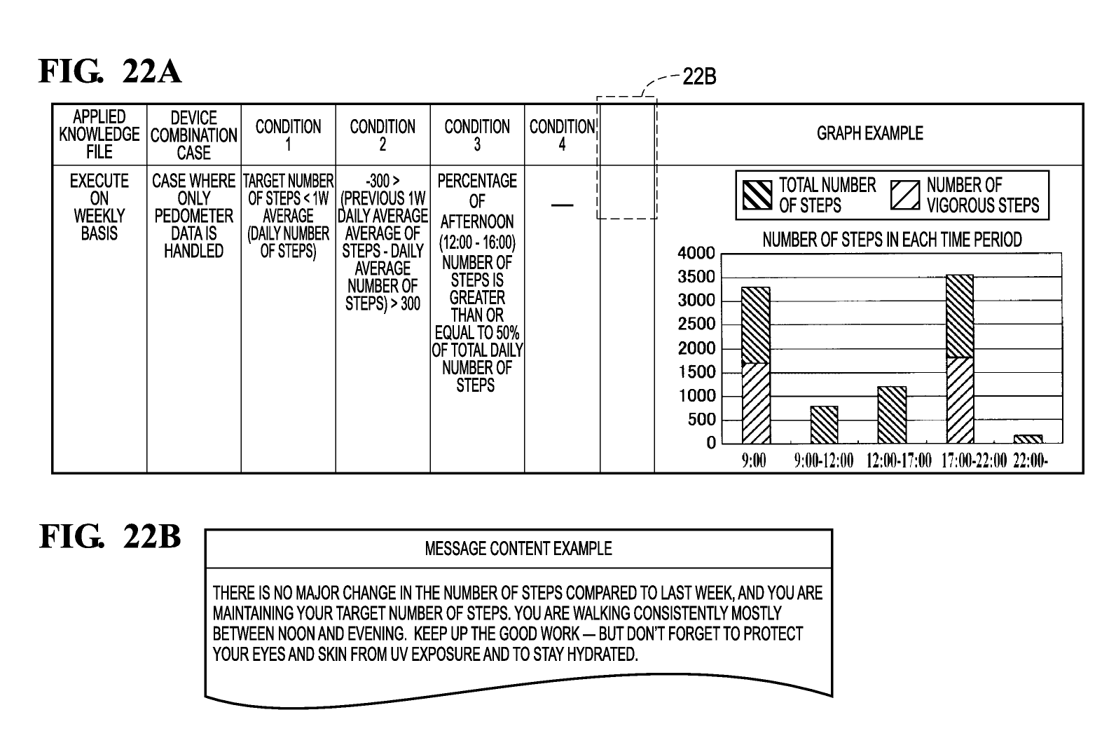

FIG. 22, meanwhile, illustrates an example of analysis details (conditions 1 through 4 of the applied rules and the outputted message 7 and graph 8) for one type of physical information collected from the pedometer 33, for the case where the analysis is executed on a “weekly basis”. In FIG. 22, an example of the content of the message is shown in greater detail.

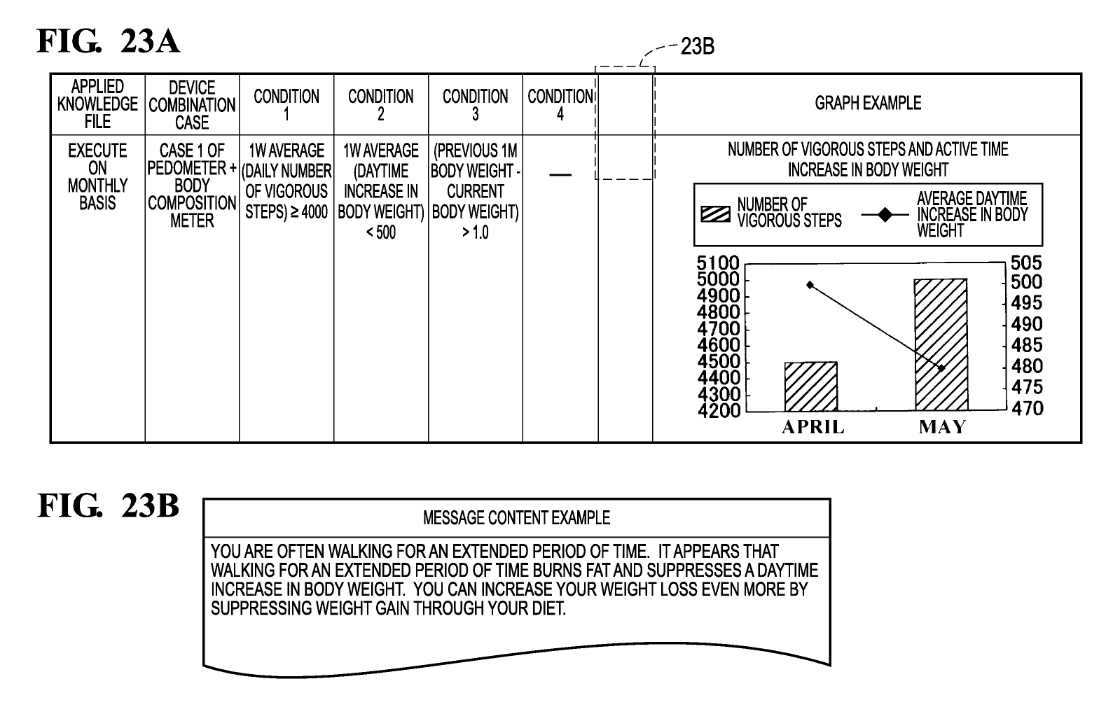

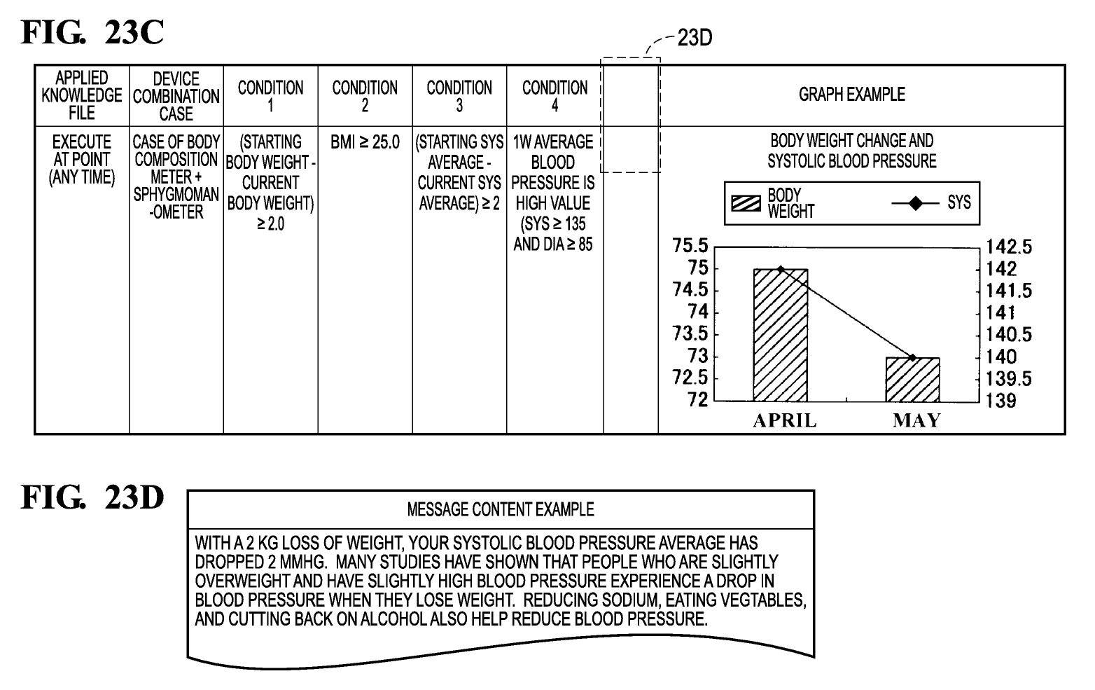

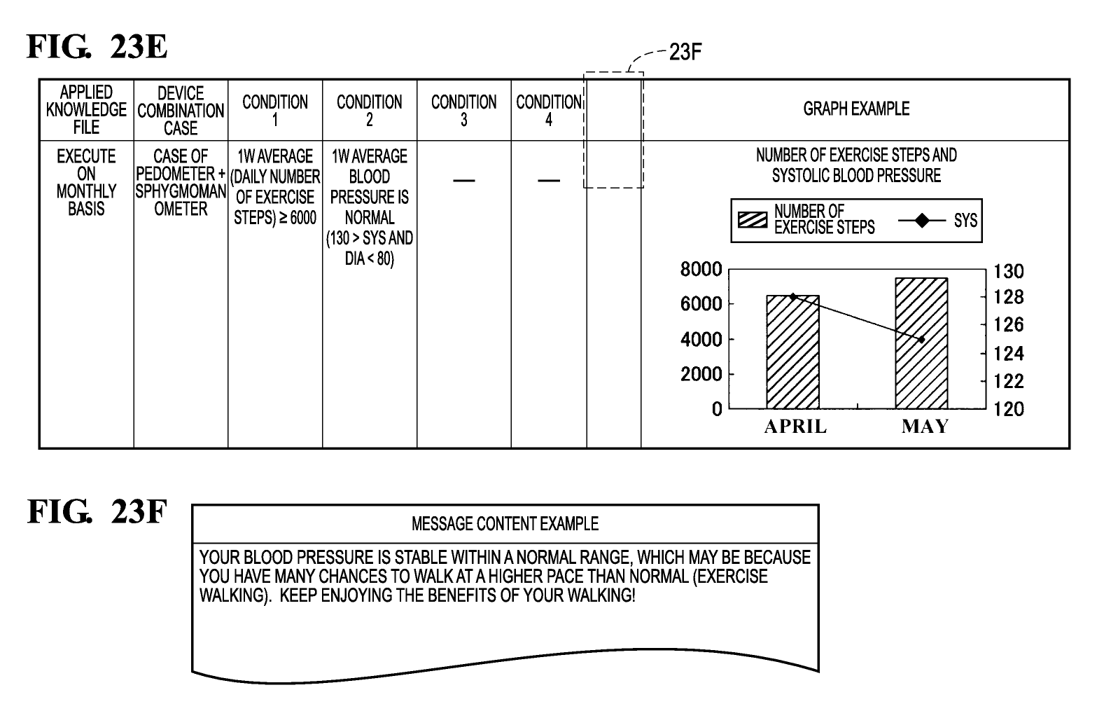

FIG. 23A illustrates an example of analysis details (conditions 1 through 4 of the applied rules and the outputted message 7 and graph 8) for two or more types of physical information collected from the pedometer 33 and the scale/body composition meter 34 on a “monthly basis”; FIG. 23B illustrates an example of analysis details (conditions 1 through 4 of the applied rules and the outputted message 7 and graph 8) for two or more types of physical information collected from the sphygmomanometer 32 and the scale/body composition meter 34 on an “immediate basis”; and FIG. 23C illustrates an example of analysis details (conditions 1 through 4 of the applied rules and the outputted message 7 and graph 8) for two or more types of physical information collected from the pedometer 33 and the sphygmomanometer 32 on a “monthly basis”. In FIGS. 23A, 23B, and 23C, examples of the content of the messages are shown in greater detail.

As shown in these drawings, the graph 8 is presented in various states, such as a polygonal line graph and a column graph.

It is possible to notify the user of a goal achievement level by analyzing two types of physical information over a comparatively short period of time (for example, one week), and provide advice for improving lifestyle patterns in order to enable the user to accomplish his or her goals by analyzing such lifestyle patterns over a comparatively long period of time (for example, two weeks, one month, or the like). Information regarding the relationship between lifestyle patterns (lifestyle habits) and various indexes may be provided for even longer periods as well.

In addition, a message 7 corresponding to points of change in the user's body weight/body composition or predetermined characteristics that have appeared (been detected) through the graph 8 as time passes can also be displayed at the same time as the graph 8 or in association therewith.

Thus the configuration assists users through advice provided at change points, when characteristics appear, and so on in order to continuously support behavior modification for health management, by analyzing healthcare device data, operational information, user data obtained from lifestyle information records and the like through the analysis of data changes (degrees of change and the like) such as reference value evaluation and changes over time on a daily, weekly, and monthly basis, the extraction of characteristics from data patterns, the analysis of correlation between data from different devices, between device data and lifestyle information, and so on. Accordingly, by automatically intervening as appropriate, an effect in which the an increased rate of continuation of the behavior modification can be achieved, because more personalized information can be provided, a sense of burden caused by operating a conversation-type information provision system can be lightened, and a sense of anticipation for the next use can be fostered in users.

Morning/Evening Body Weight Management Program

The health support system according to the present embodiment provides a weight loss/body weight control support system in which morning/evening body weights measured by a scale or a scale/body composition meter 34 are sent to the server device 1, and the server device 1 outputs the message 7 and the graph 8 using the body weight that increases from morning to evening and the body weight that decreases from evening to morning as an index for weight loss.

When the user selects “morning/evening diet” in the menu screen shown in FIG. 18, analysis for weight loss/body weight control support is carried out using a morning/evening body weight difference, and the message 7 and graph 8 are provided. At this time, the user ID and a request for “body weight/body composition data analysis” (called a “morning/evening diet request” hereinafter) are sent from the information terminal 22 to the server device 1. When the “morning/evening diet” request is made, the morning/evening body weight calculation unit 4B of the calculation unit 4A is started up.

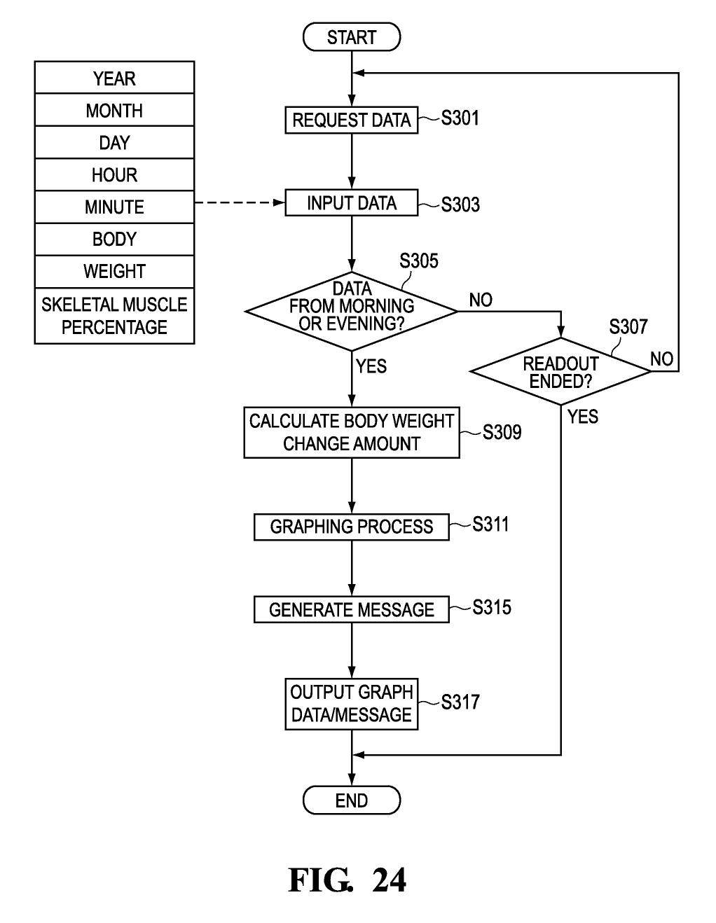

FIG. 24 is a flowchart illustrating a process carried out by the server device 1 for the morning/evening diet program. When the morning/evening diet request is received, the morning/evening diet program is started, and the engine startup unit 151 starts up the engine unit 4.

First, when the control unit 15 inputs the user ID and the morning/evening diet request (step S301), the data extraction unit 156 searches the body composition meter database DB4 of the data accumulation unit 2 based on the user ID and the morning/evening diet request, and reads out, from the data accumulation unit 2, body weight data measured over a set period in the past along with associated skeletal muscle percentage and measurement time data (step S303).

The data extraction unit 156 determines whether or not the measurement time of the read-out data indicates a morning time period (from 5:00 to 10:00) or an evening time period (from 20:00 to 5:00 the next day) (step S305), and outputs only data measured during that time period to the input data setting unit 152. In this manner, the readout of all data measured in a set period in the past, and the determination of the time period, are carried out (steps S301 to S307).

When it has been determined that the inputted data is morning time period or evening time period data (YES in step S305), the inputted data set 6A is generated by the input data setting unit 152 using a variable definition information 5A for the morning/evening diet program. The morning/evening body weight calculation unit 413 carries out a calculation process based on the variable values in the inputted data set 6A and a morning/evening body weight change amount calculation formula in the preliminary calculation formula information 5B for the morning/evening diet program (step S309). The result of the calculation is outputted to the rule execution unit 4C, rules in a message generation rule group 5C for the morning/evening diet program are executed, and a process for generating the graph 8 is carried out by the graph creation unit 154 (step S311). The data of the message 7 is then generated (step S315). Destination information is added to the generated graph 8 and message 7 through the communication unit 10, after which the graph 8 and the message 7 are sent to the information terminal 22 and displayed in the display unit 15A (step S317).

The procedures for creating the graph 8 and the message 7 are essentially the same as those described above, and thus detailed descriptions thereof will be omitted here.

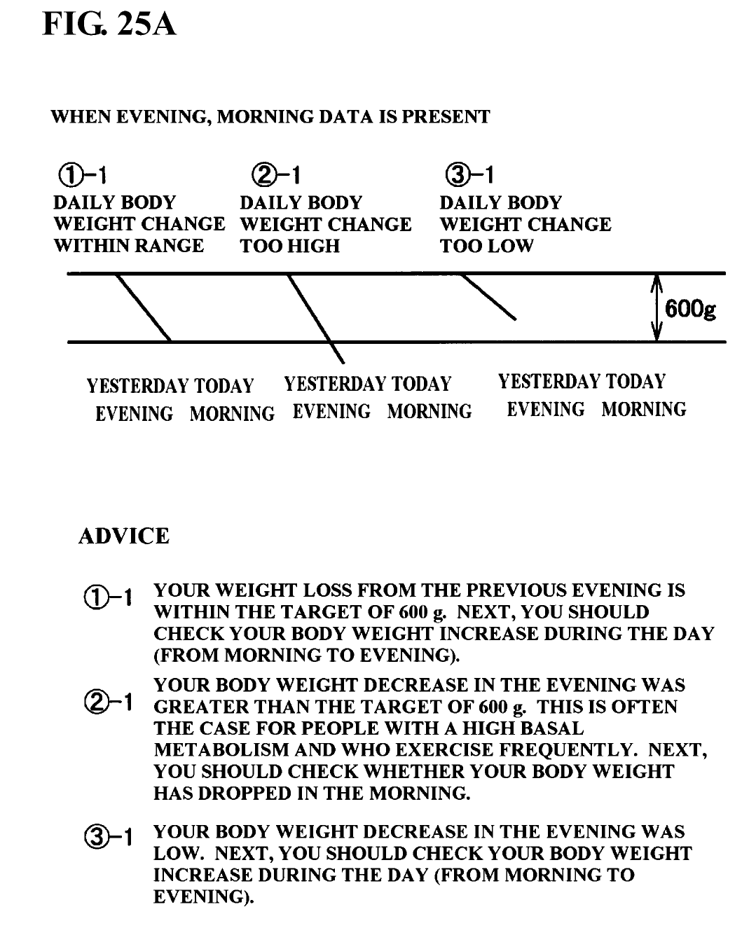

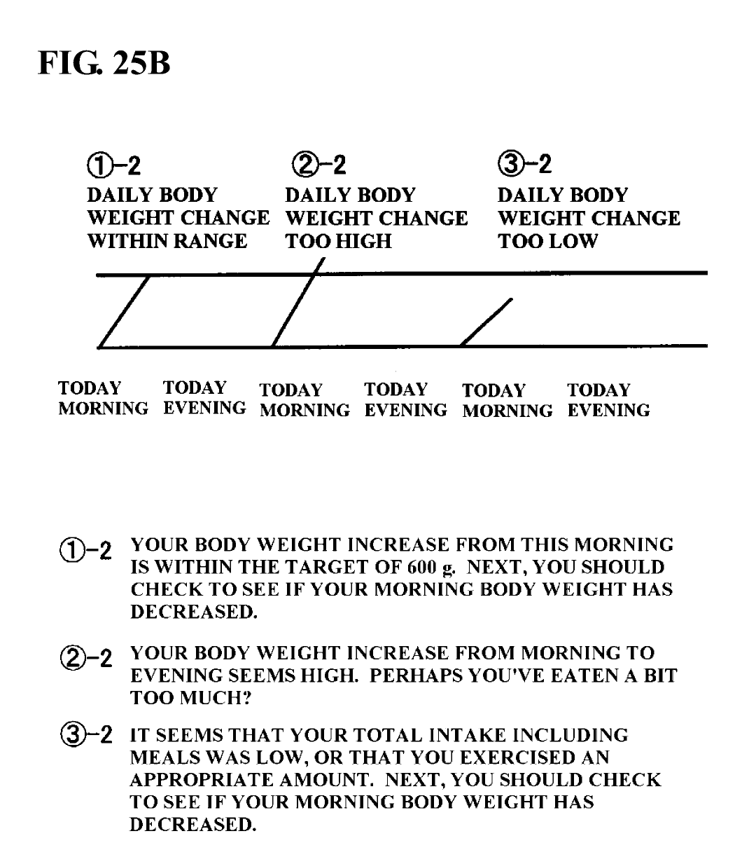

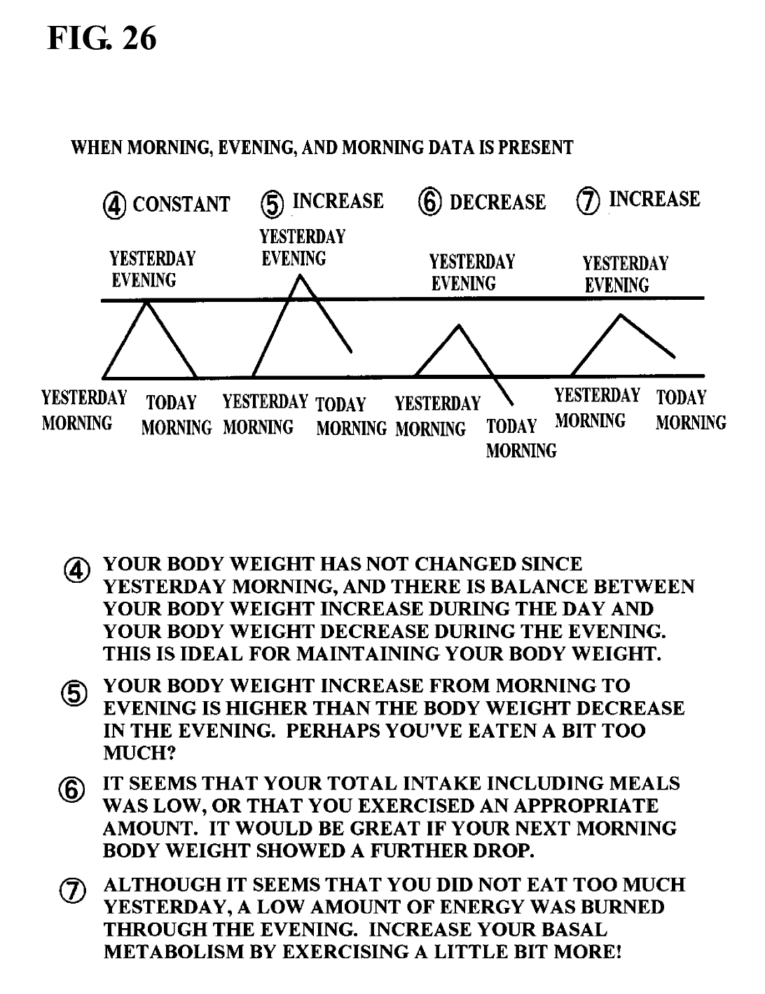

FIGS. 25A, 25B, and 26 illustrate examples of the display of the graph 8 and the message 7, where a reference value is provided for the morning/evening body weight change amount, and a graph 8 that compares the measured body weight change amount with the reference value is displayed in association with the message 7 (advice) based on the result of that comparison. FIGS. 25A and 2513 illustrate an example of a result of comparing morning measurement data with evening measurement data, whereas FIG. 26 illustrates an example of comparing morning measurement data, evening measurement data, and morning measurement data. The user can be notified of the goal achievement level through such a daily analysis.

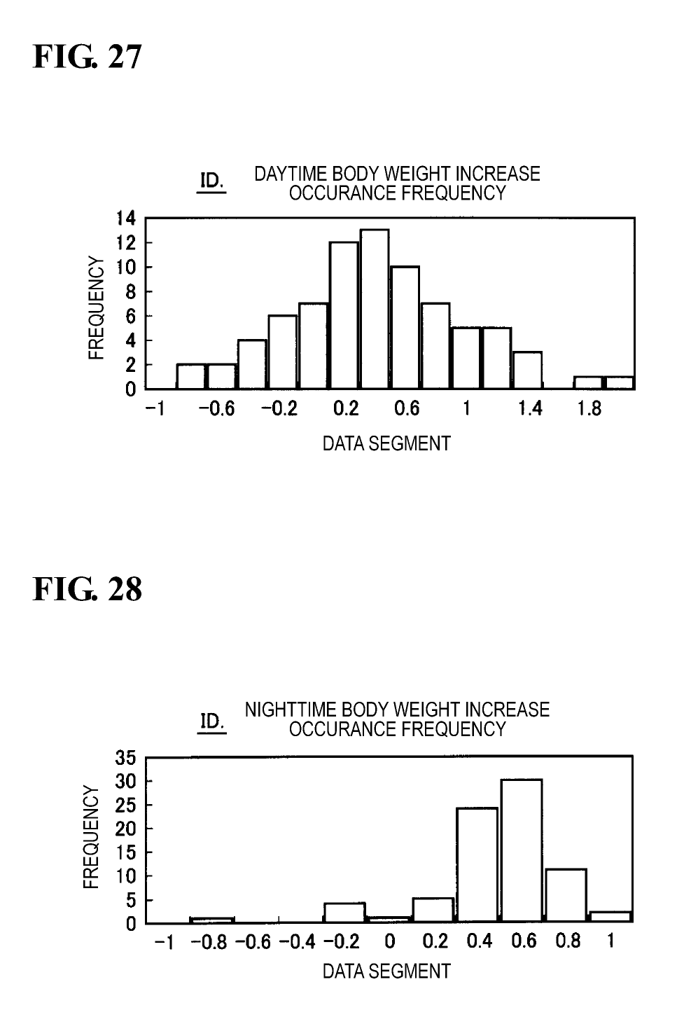

FIGS. 27 and 28 illustrate a daytime weight increase occurrence frequency and a nighttime weight increase occurrence frequency using a histogram, as an example of the display of the graph 8, which enables the user to know a mode value, variations, and so on. The morning/evening body weight calculation unit 4B calculates the nighttime weight increase by subtracting this morning's body weight from last evening's body weight, and calculates the daytime weight increase by subtracting this morning's body weight from this evening's body weight. Meanwhile, a daily body weight change can be calculated by subtracting this morning's body weight from yesterday morning's body weight, a variation between the morning/evening body weight change amount can be calculated, and the results can be displayed as a graph.

Variations occur in body weight gain due to variations in measurement times, how the user hydrates, unevenness in food requirements and mealtimes, and so on, and thus by checking the graph 8 shown in FIGS. 27 and 28, the user can reduce those variations as much as possible to make it easier to create each day's goals.

In addition, it is possible for the user to know his or her current average active time (morning-evening) body weight increase amount. A mode value can be obtained from a body weight increase amount distribution. It is also possible for the user to know his or her current average sleeping time (evening-morning) body weight decrease amount, and obtain a mode value from a body weight decrease amount distribution.

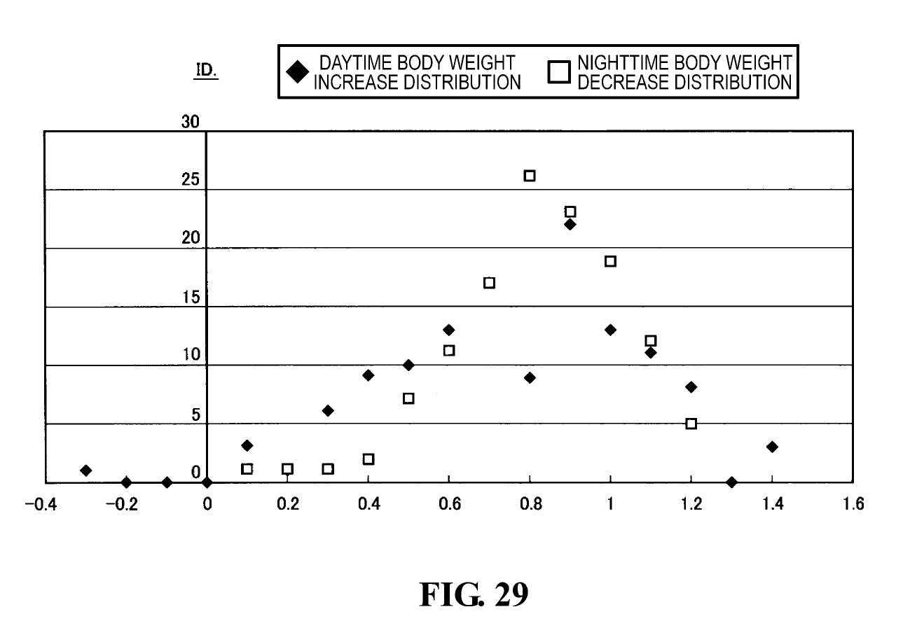

FIG. 29 illustrates an example of the display of another graph. In FIG. 29, the daytime weight increases and nighttime weight decreases in a set period are added according to specified data segments, and the frequencies thereof are indicated as a histogram.

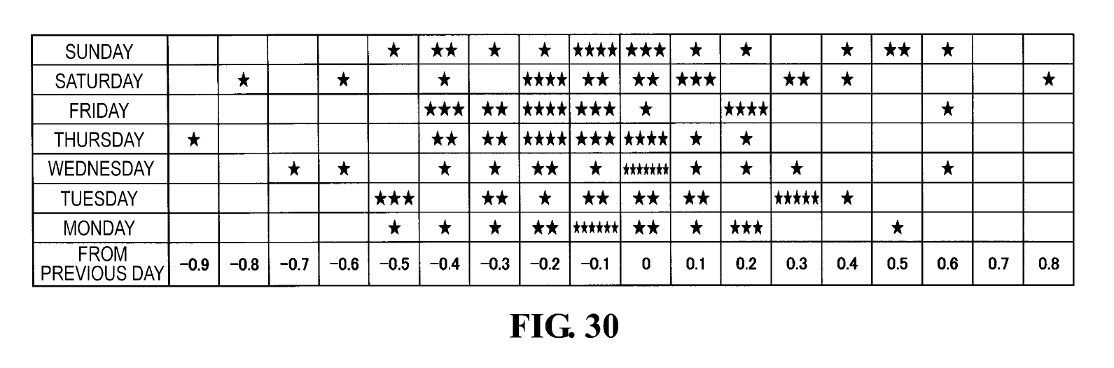

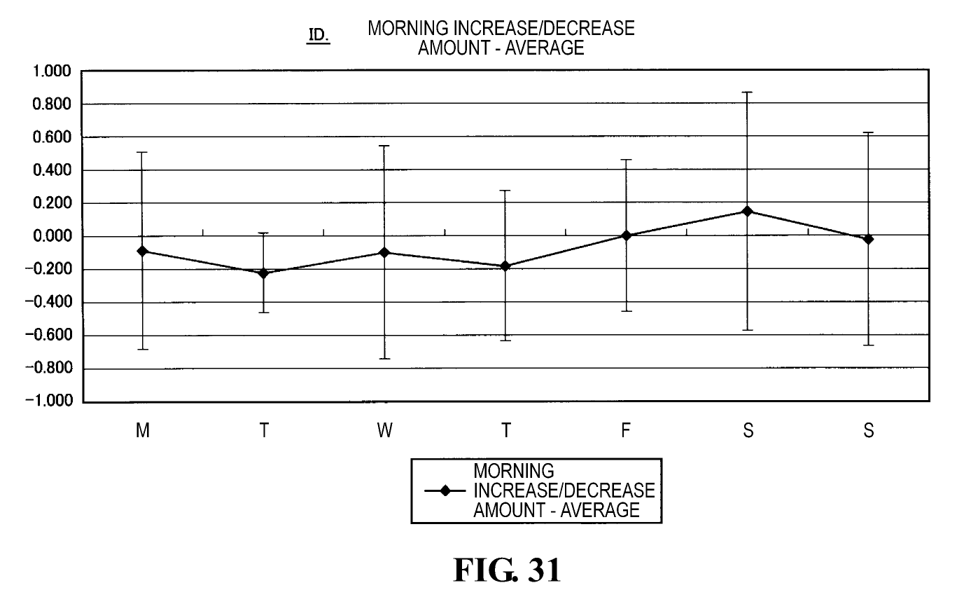

FIG. 30 illustrates the graph 8, in which the body weight change amounts from the previous day in a set period are added according to specified data segments, and the frequencies thereof are distributed according to the days of the week. FIG. 31 illustrates the graph 8, in which the maximum value, minimum value, an average value of the body weight change amounts from the previous day in a set period are indicated for each day of the week.

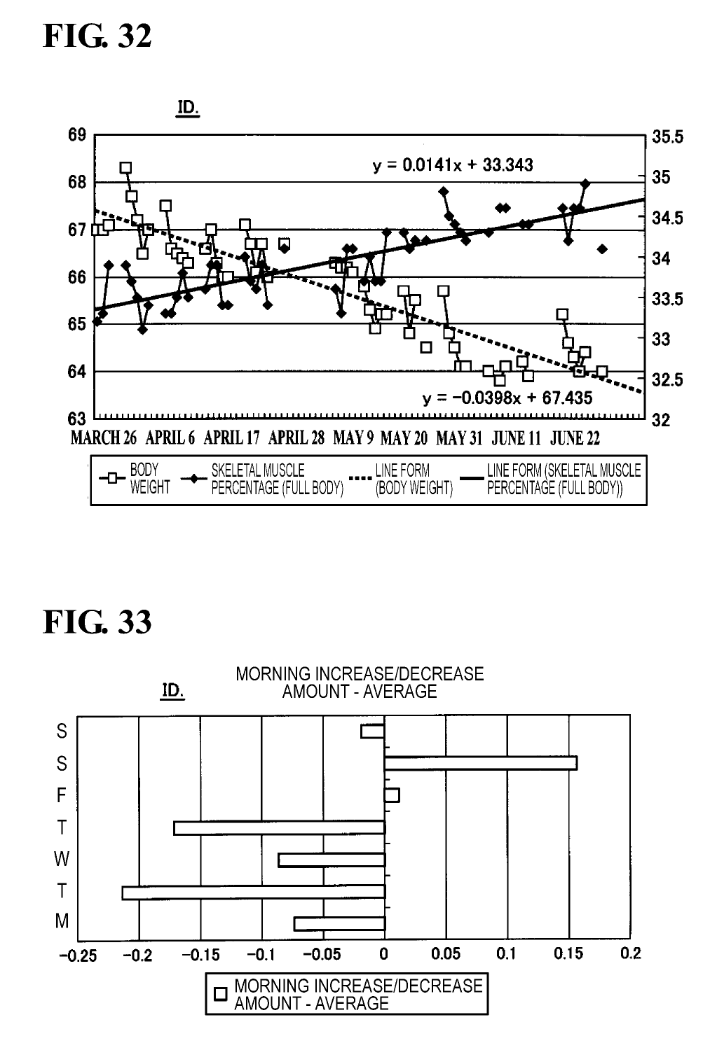

FIG. 32 illustrates changes in the measured values of a user's body weight and skeletal muscle percentage (a polygonal line graph), and approximated straight lines and straight line formulas for those changes are illustrated.

FIG. 33 illustrates an average increase/decrease amount for a user's morning body weight, on a day-of-the-week basis. With the graph 8 in FIG. 33, the user's attention is called to his or her lifestyle pattern on a weekly basis. For example, the user can be motivated to improve the way he or she spends his or her days off.

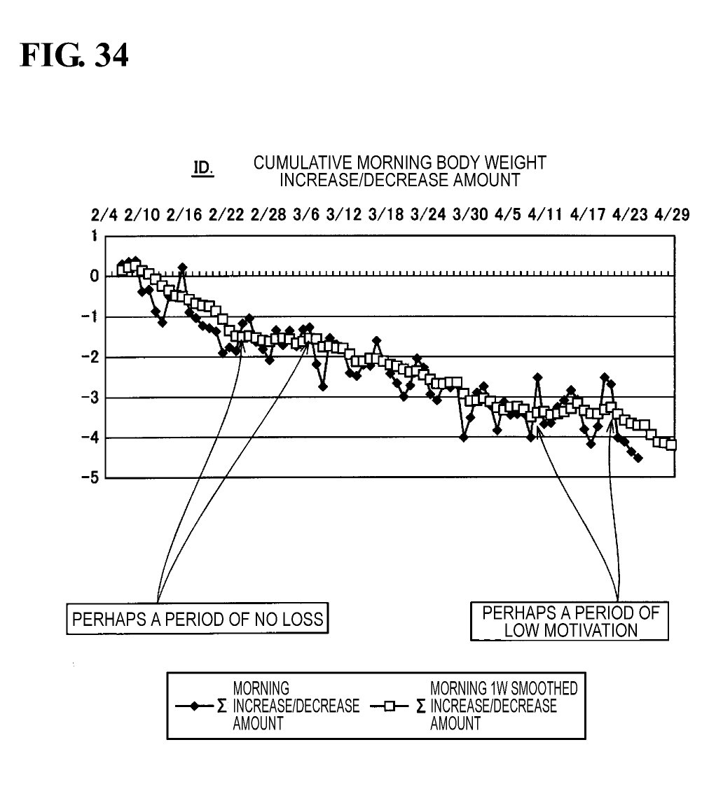

In FIG. 34, the cumulative value for a user's morning body weight increase/decrease amount is graphed along a time axis. The graph 8 in FIG. 34 also illustrates the average amount of weight increase/decrease on a weekly basis. The results of continuing a diet for a long period of time (three months or the like) can be checked. If the user succeeds in losing body weight, it is also possible that his or her blood pressure will approach a normal value, and thus the message 7 may be displayed so as to prompt the user to measure and confirm his or her blood pressure using the sphygmomanometer 32.

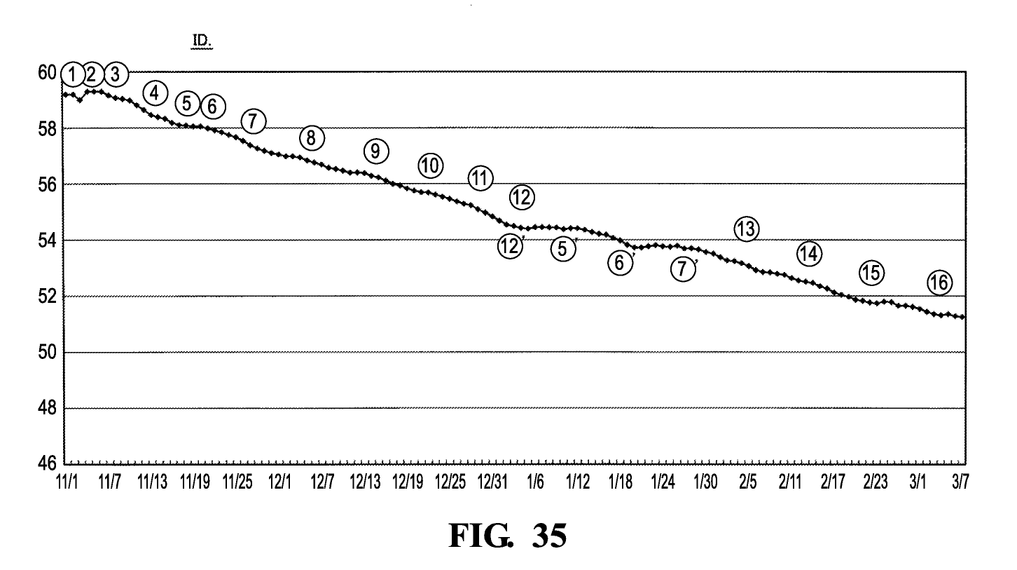

FIG. 35 illustrates the graph 8, in which calculation values that smooth (that is, find a running average) the past week's worth of body weight measurement data are plotted in time series, in the case where the user is attempting to continue a diet over a long period of time. In addition, this graph 8 also displays message numbers (the numerical values 1 through 16 in the circles shown in FIG. 35) that correspond to the timings at which points of change appear in the user's body weight or at which characteristics appear (characteristics are detected). When the user specifies the numerical value of a message number by clicking that number or the like using the operation unit 204, the message 7 associated with that message number is displayed.

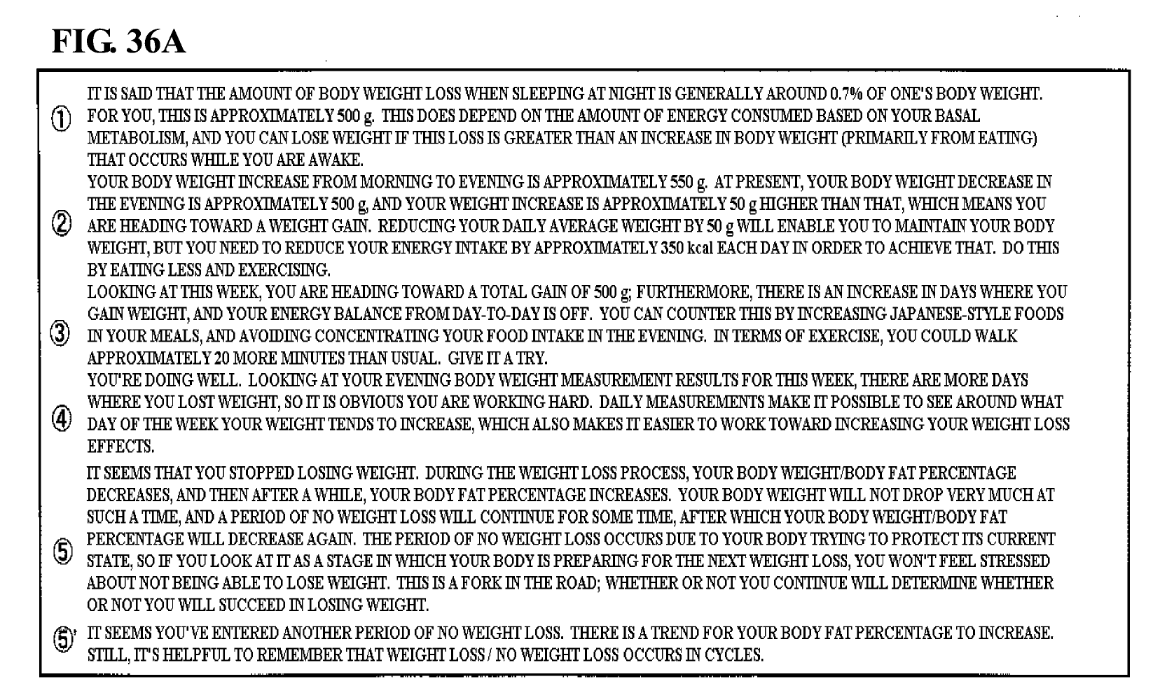

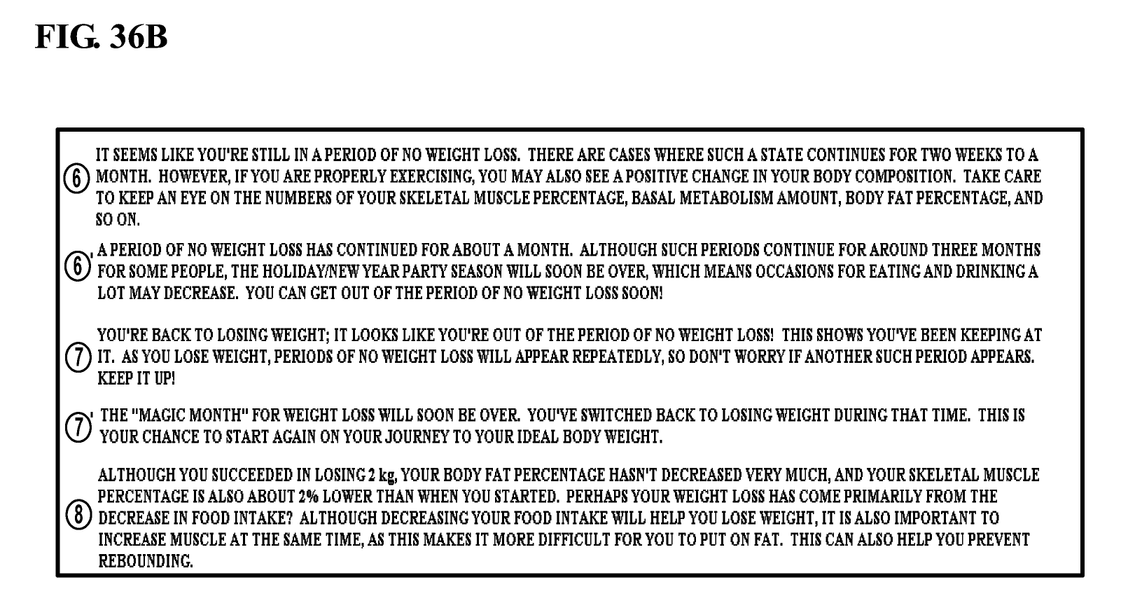

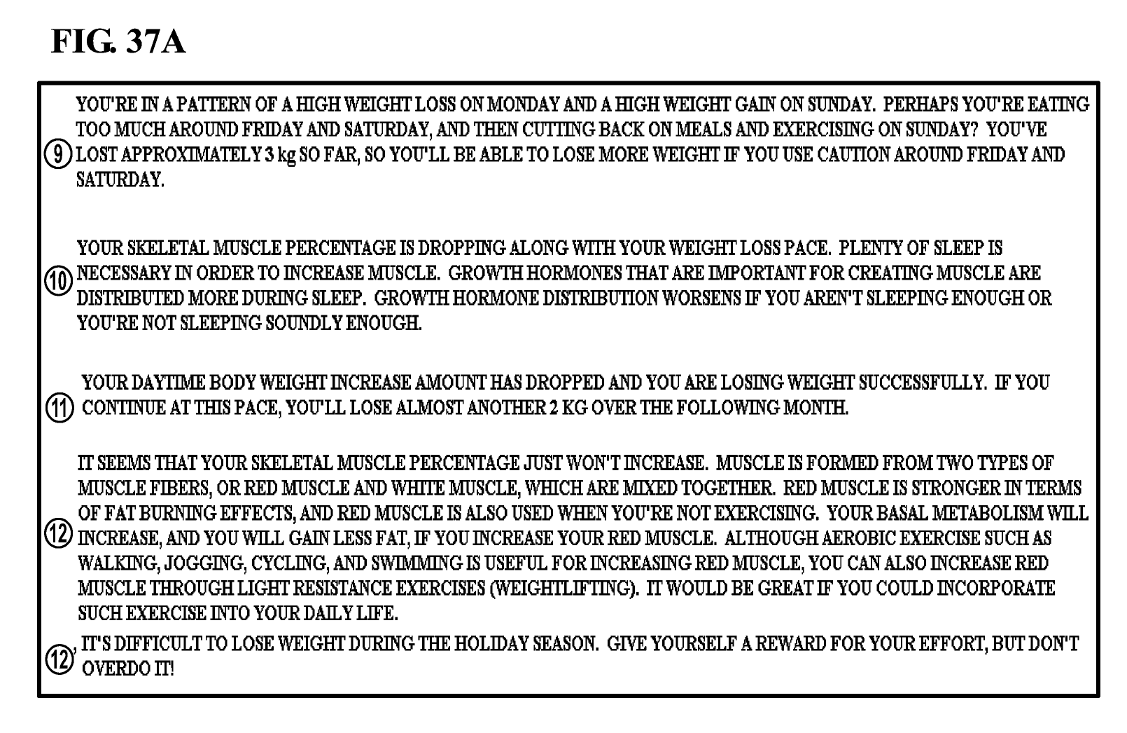

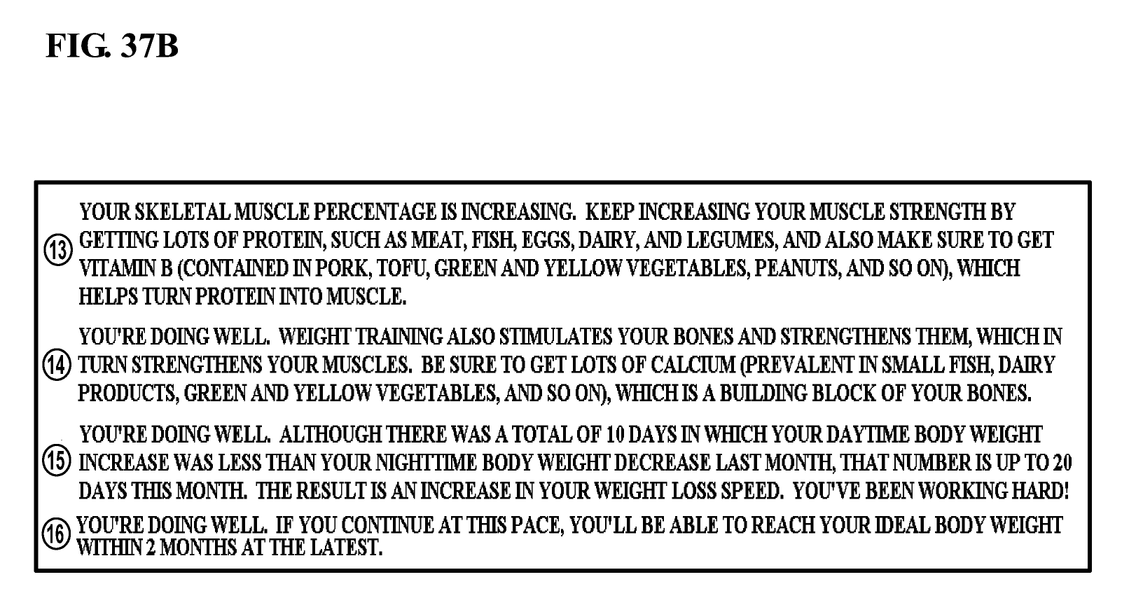

The messages 7 that correspond to the respective message numbers that are displayed are shown in the lists in FIGS. 36A and 368 and FIGS. 37A and 37B. Each message 7 presents advice, encouragement, or the like related to the points of change in body weight, appearances of characteristics, and so on.

By analyzing lifestyle patterns through the analysis of body weight data from a comparatively long period of time and generating and presenting the message 7 and the graph 8 based on the results of that analysis, it is possible to provide advice to the user for improving his or her lifestyle patterns in order to enable him or her to achieve his or her goals.

The configuration of the system for supporting weight loss, body weight control, or the like is configured so that frequency distributions are created for the “body weight that increases from morning to evening” and the “body weight that decreases from evening to morning” based on day-of-the-week data from a set period, and mode values, variation values, and so on are calculated for the respective distributions and are displayed as graphs, numerical values, or the like. Accordingly, the user can know a target for his or her daily caloric intake and calorie consumption and the user can look back on the relationship between his or her lifestyle patterns and body weight over a comparatively short amount of time, such as one week, which makes it possible to achieve an effect in which the user is motivated to lose weight or control his or her body weight, the user experiences an increased rate of continuation of the behavior modification, and so on.

Although the program for the health management support system that includes morning/evening body weight management is described as being executed by the server device 1, it should be noted that in the case where the environment shown in FIG. 2 is configured in the information terminal 22, the health management support device corresponds to the information terminal 22, and the information terminal 22 can provide the message 7 and the graph 8 via the display unit 205 by executing the processes.

In addition, if the hardware functionality of the scale/body composition meter 34, which serves as a healthcare device, is expanded, the environment shown in FIG. 2 can also be configured therein. In that case, the health management support device corresponds to the scale/body composition meter 34, and the message 7 and graph 8 can be provided via a display unit 154A.

Although the present embodiment illustrates an example in which analysis is carried out based on physical information collected from a healthcare device, the data that serves as the base is not limited to physical information. For example, operational information such as the frequency of use of the healthcare device, lifestyle information (sleep time, whether or not the user is engaged in shift work, or the like), and so on may be collected, and these pieces of information may be analyzed in combination with each other.

In addition, it is known that one's health is related to the surrounding climate (weather), and thus weather information may be collected from a database in an external organization, and the information may be analyzed in combination with the weather information.

Furthermore, a user's health examination information may be collected from a database in a hospital, a clinic, or the like, and the information may be analyzed in combination with the health examination information.

Note that the method for analyzing information and presenting health management advice based on a result of the analysis according to the present embodiment can also be provided as a program. Such a program can also be recorded on a computer-readable non-transitory recording medium, such as an optical medium including CD-ROM (compact disc-ROM), a memory card, or the like, and provided as a program product. Further still, the program can also be downloaded via a network, and can be provided in such form as a program.

Note that the program according to the present invention may execute processing by calling, in a predetermined arrangement and at a predetermined timing, the necessary program modules from among the modules provided as part of an operating system (OS) of a computer. In this case, the stated modules are not included in the program itself, and the processing is executed in cooperation with the OS. Such a program that does not include modules in this manner can also fall within the scope of the program according to the present invention.

In addition, the program according to the present invention may be provided having been incorporated into a part of another program. In such a case as well, modules included in the stated other program are not included within the program itself, and the processing is executed in cooperation with the other program. Such a program that is incorporated into another program can also fall within the scope of the program according to the present invention.

The program product that is provided is installed in a program storage unit such as a hard disk and executed. Note that the program product includes the program itself and the storage medium in which the program is stored.

In this manner, the embodiments and variations disclosed herein are to be understood in all ways as exemplary and in no ways limiting. The technical scope of the present invention is defined by the appended claims, and all variations that fall within the meaning and range of equivalency of the claims are intended to be embraced therein.

### REFERENCE SIGNS LIST

1 server device
2 data accumulation unit
4 engine unit
5 knowledge file group
6 graph creation unit
7 message
8 graph
15 control unit
21-23 information terminal
34 scale/body composition meter
51-53 communication path
4A calculation unit
4B morning/evening body weight calculation unit
4C rule execution unit
4D graph creation request unit
6A inputted data set
5D message file
5E graph creation guideline information

1. A health management support device comprising:
a receiving unit that receives two or more types of physical information measured for a user along with measurement time data;
an analyzing unit for analyzing the relationship between the received two or more types of physical information in accordance with a predetermined rule;
an advice generating unit that generates advice based on a result of the analysis;
an advice output unit that outputs the generated advice;
a unit that receives body weight data of the user along with measurement time data;
a determination unit that determines, based on the measurement time data, whether or not the body weight data is body weight data measured during a morning time period or an evening time period; and
a calculation unit that calculates, according to time series, a morning/evening body weight change amount over a set period for the body weight data determined by the determination unit to have been measured during the morning time period or the evening time period,
wherein a “body weight that increases from morning to evening” and a “body weight that decreases from evening to morning” are outputted as a graph based on the morning/evening body weight change amount during the set period calculated by the calculation unit;
a knowledge file that stores the predetermined rule; and
an engine unit for executing the analysis, and
wherein the advice generating unit generates the advice for notifying the user of a goal achievement level by analyzing the two or more types of physical information measured in a first predetermined period.

2. The health management support device according to claim 1,
wherein the advice generating unit generates the advice for enabling the user to achieve a goal by analyzing the two or more types of physical information measured in a second predetermined period.

3. The health management support device according to claim 1,
wherein the analyzing unit analyzes changes over time in the two or more types of physical information in each of predetermined measurement periods.

4. The health management support device according to claim 3,
wherein the predetermined measurement period includes a daily basis, a weekly basis, or a monthly basis.

5. The health management support device according to claim 4,
wherein the advice generating unit generates advice corresponding to points in the changes over time analyzed by the analyzing unit.

6. The health management support device according to claim 4,
wherein the advice generating unit generates advice corresponding to a predetermined characteristic detected over time and analyzed by the analyzing unit.

7. The health management support device according to claim 1,
wherein the analyzing unit analyzes, in accordance with a predetermined rule, the two or more types of physical information and a different type of information than the physical information for a relationship between the two or more types of physical information and the different type of information than the physical information.

8. The health management support device according to claim 1,
wherein the calculation unit totals the morning/evening body weight change amount for each day of the week.

9. The health management support device according to claim 1,
wherein the calculation unit calculates a variation in the morning/evening body weight change amount.

10. The health management support device according to claim 1,
wherein a frequency distribution is created for a “body weight that increases from morning to evening” and a “body weight that decreases from evening to morning” based on the morning/evening body weight change amount that is based on the body weight data measured during the set period, and the frequency distribution is displayed as a graph, for each day of the week.

11. A health management support system comprising a server device and an information terminal that sends two or more types of physical information measured for a user to the server device along with measurement time data and outputs information received from the server device,
wherein the server device includes:
a receiving unit that receives, from the information terminal, the two or more types of physical information along with the measurement time data;
an analyzing unit for analyzing the relationship between the received two or more types of physical information in accordance with a predetermined rule;
an advice generating unit that generates advice based on a result of the analysis;
a sending unit that sends the generated advice to the information terminal;
a means for receiving body weight data of the user along with measurement time data;
a determination unit that determines, based on the measurement time data, whether or not the body weight data is body weight data measured during a morning time period or an evening time period; and
a calculation unit that calculates, according to time series, a morning/evening body weight change amount over a set period for the body weight data determined by the determination unit to have been measured during the morning time period or the evening time period,
wherein a “body weight that increases from morning to evening” and a “body weight that decreases from evening to morning” are outputted as a graph based on the morning/evening body weight change amount during the set period calculated by the calculation unit;
wherein the analyzing unit includes:
a knowledge file that stores the predetermined rule; and
an engine unit for executing the analysis, and
wherein the advice generating unit generates the advice for notifying the user of a goal achievement level by analyzing the two or more types of physical information measured in a first predetermined period.

12. The health management support system according to claim 11,
wherein the advice generating unit generates the advice for enabling the user to achieve a goal by analyzing the two or more types of physical information measured in a second predetermined period.

13. The health management support system according to claim 11, further comprising:
one or more healthcare devices for measuring the two or more types of physical information for the user.

14. A health management support program that processes two or more types of physical information measured for a user, the program causing a computer to execute:
a step of receiving the two or more types of physical information along with measurement time data;
a step of analyzing the relationship between the received two or more types of physical information in accordance with a predetermined rule;
a step of generating advice based on a result of the analysis;
a step of outputting the generated advice;
a step of receiving body weight data of the user along with measurement time data;
a step of determining, based on the measurement time data, whether or not the body weight data is body weight data measured during a morning time period or an evening time period; and
a step of calculating, according to time series, a morning/evening body weight change amount over a set period for the body weight data determined in the step of determining to have been measured during the morning time period or the evening time period,
wherein a “body weight that increases from morning to evening” and a “body weight that decreases from evening to morning” are outputted as a graph based on the morning/evening body weight change amount during the set period calculated in the step of calculating;
wherein the step of analyzing includes a step of referring to a knowledge file that stores the predetermined rule and executing the analysis; and
the step of generating the advice generates the advice for notifying the user of a goal achievement level by analyzing the two or more types of physical information measured in a first predetermined period.
